(19) United States
(12) Patent Application Publication
(10) Pub. No.: US 2026/0301917 A1
(43) Pub. Date: Oct. 1, 2026

(54) INFORMATION PROCESSING DEVICE, INFORMATION PROCESSING METHOD, AND PROGRAM

(71) Applicant: SONY GROUP CORPORATION, TOKYO (JP)

(72) Inventors: SHINICHI TERAGUCHI, TOKYO (JP); KAREN MURATA, TOKYO (JP)

(21) Appl. No.: 19/479,484

(22) PCT Filed: Apr. 25, 2024
(86) PCT No.: PCT/JP2024/016192

§ 371 Date: Oct. 28, 2025

(30) Foreign Application Priority Data
May 11, 2023 (JP) 2023-078372

(51) Int. Cl. G16H 20/70; A61M 21/02; G16H 40/67

(52) U.S. Cl. CPC G16H 20/70; A61M 21/02; G16H 40/67

(57) ABSTRACT

The present technology relates to an information processing device, an information processing method, and a program capable of effectively evoking a sense of connection in a user.

The mindfulness control section performs control in such a way as to provide a first stimulus for an experiencer in a mindfulness and relaxation mode, which is an operation mode for bringing the experiencer into a mindful state and a relaxed state. The connection evoking control section provides a second stimulus for the experiencer in a connection evoking mode, which is an operation mode for evoking a sense of connection in the experiencer, on the basis of recalled person information, which is information acquired from a recalled person, who evokes the sense of connection. The provision of the first stimulus is initiated prior to the provision of the second stimulus. The present technology can be applied to, for example, a providing device and the like.

### TECHNICAL FIELD

The present technology relates to an information processing device, an information processing method, and a program, and more particularly, to an information processing device, an information processing method, and a program capable of effectively evoking a sense of connection in a user.

### BACKGROUND ART

In recent years, with the rise of nuclear families and the advent of the 100-year life era, there is an increasing possibility that people may experience loneliness more frequently or for longer periods, especially after parting with a partner. A feeling of loneliness and social isolation can elevate the levels of stress hormones and inflammation, thereby increasing the risk of conditions such as heart disease, type 2 diabetes, dementia, and suicide.

The social isolation refers to a state in which there is little connection or interaction with society. The feeling of loneliness is a subjective recognition of social isolation, and occurs when a level of an individual's desire for connection and interaction with society is higher than an actual level.

For example, even a person who is socially isolated does not feel lonely in a case where the person prefers to be on his/her own, and even a person who is not socially isolated feels lonely in a case where the person desires further connection or interaction with society. Therefore, it is difficult to externally determine occurrence of a feeling of loneliness, and the occurrence of a feeling of loneliness depends more on the subjective recognition of a person himself, that is, the state of mind than the situation. Therefore, in order for a person to eliminate a feeling of loneliness, it is effective for the person to recognize or recall a feeling of being connected to a person or an object required by the person (hereinafter referred to as a sense of connection).

On the other hand, we now live in an era of volatility, uncertainty, complexity, and ambiguity (VUCA), a time that is difficult to predict, and social networking services (SNSs) have negative aspects, such as mechanisms that excessively stimulate the desire for approval and those that induce dependency. As a result, the number of people who feel lonely or hard to live and the number of people who cannot have confidence in themselves are increasing, and as a result, the number of people who suffer from a mental illness due to stress or trauma is increasing. For example, the proportion of people in Japan suffering from depression or depressive symptoms was 7.9% in 2013, but increased to 17.3% in 2020, approximately doubling over that period.

However, it is difficult to immediately improve the surrounding situation and system and eliminate the factors of stress and trauma. Therefore, it is important to cultivate a state of mind that is not crushed by the current situation. Therefore, transforming the human mind itself and enhancing inner sustainability, such as resilience, has gained new value and significance, and the importance of methods to achieve this is increasing.

Note that resilience is a non-cognitive ability to overcome an adverse situation and grow on the basis of the situation. For example, resilience enables a person to recover from a shock and adapt to an adverse situation when facing the adverse situation, such as a harsh environment, a stressful situation, or a traumatic experience.

In order to enhance inner sustainability, self-compassion is effective, and there is a deep relationship between resilience, a sense of connection, and self-compassion (see, for example, Non-Patent Document 1).

As described above, evoking a sense of connection is important not only for eliminating a feeling of loneliness but also for improving inner sustainability.

Meanwhile, there is a biological information sharing system that detects biological information regarding a subject and that performs an operation of expressing the biological information in a predetermined manner (see, for example, Patent Document 1).

### CITATION LIST

### Patent Document

Non-Patent Document 1: Kohki Arimitsu, “DIAMOND Harvard Business Review Paper, Self-Composition: How to stay the best”, published by DIAMOND, Inc., Apr. 10, 2019

Patent Document 1: Japanese Patent Application Laid-Open No. 2009-15449

### SUMMARY OF THE INVENTION

### Problems to be Solved by the Invention

However, a method for effectively evoking a sense of connection in a user has not been devised.

The present technology has been made in view of such circumstances, and an object thereof is to effectively evoke a sense of connection in a user.

### Solutions to Problems

An information processing device or a program according to an aspect of the present technology is an information processing device including a mindfulness control section that performs control in such a way as to provide a first stimulus for a user in a mindfulness mode, which is an operation mode for bringing the user into a mindful state, and a connection evoking control section that performs control in a connection evoking mode, which is an operation mode for evoking a sense of connection in the user, in such a way as to provide a second stimulus for the user on the basis of recalled person information, which is information acquired from a recalled person, who evokes the sense of connection, in which the provision of the first stimulus is initiated prior to the provision of the second stimulus, or a program causing a computer to function as the information processing device.

An information processing method according to an aspect of the present technology is an information processing method performed by an information processing device, the information processing method including a mindfulness control step of performing control in such a way as to provide a first stimulus for a user in a mindfulness mode, which is an operation mode for bringing the user into a mindful state, and a connection evoking control step of performing control in a connection evoking mode, which is an operation mode for evoking a sense of connection in the user, in such a way as to provide a second stimulus for the user on the basis of recalled person information, which is information acquired from a recalled person, who evokes the sense of connection, in which the provision of the first stimulus is initiated prior to the provision of the second stimulus.

In the aspect of the present technology, control is performed in the mindfulness mode, which is an operation mode for bringing the user into the mindful state, in such a way as to provide the first stimulus for the user, and control is performed in the connection evoking mode, which is the operation mode for evoking the sense of connection in the user, in such a way as to provide a second stimulus for the user on the basis of recalled person information, which is information acquired from a recalled person, who evokes the sense of connection. The provision of the first stimulus is initiated prior to the provision of the second stimulus.

### BRIEF DESCRIPTION OF DRAWINGS

FIG. 1 is a block diagram illustrating a configuration example of an embodiment of an experience system to which the present technology is applied.

FIG. 2 is a block diagram illustrating a configuration example of an acquisition device.

FIG. 3 is a block diagram illustrating a configuration example of a providing device.

FIG. 4 is a diagram illustrating a configuration example of a recalled person information table.

FIG. 5 is a diagram illustrating an example of information registered in the recalled person table.

FIG. 6 is a top view illustrating an external configuration example of a part of the providing device.

FIG. 7 is a block diagram illustrating a configuration example of a control unit.

FIG. 8 is a diagram illustrating order of operation modes.

FIG. 9 is a diagram illustrating an oxytocin enhancement loop.

FIG. 10 is a table illustrating an example of physical stimuli in an active mode.

FIG. 11 is a table illustrating an example of cognitive stimuli in the active mode.

FIG. 12 is a diagram illustrating an example of how stimuli are provided.

FIG. 13 is a diagram illustrating a configuration example of an experiencer information table.

FIG. 14 is a diagram illustrating an example of information registered in an experiencer table.

FIG. 15 is a table illustrating an example of changes to stimuli in a mindfulness and relaxation mode based on a state estimation index.

FIG. 16 is a flowchart illustrating a transmission process.

FIG. 17 is a flowchart illustrating a control process.

FIG. 18 is a flowchart illustrating a registration process.

FIG. 19 is a block diagram illustrating a configuration example of hardware of a computer.

### MODE FOR CARRYING OUT THE INVENTION

A mode for carrying out the present technology (hereinafter referred to as an embodiment) will be described hereinafter.

Note that the description will be given in the following order.
1. Embodiment (Experience System)
2. Computer

### 1. Embodiment

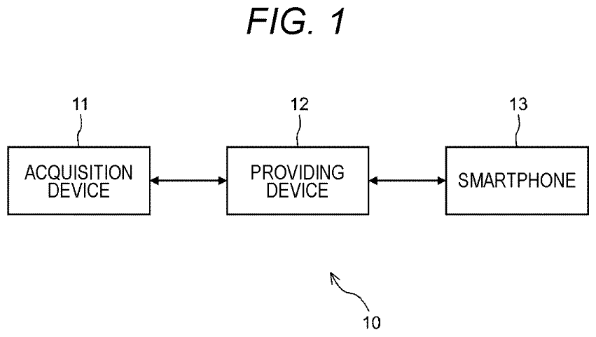

FIG. 1 is a block diagram illustrating a configuration example of an embodiment of an experience system to which the present technology is applied.

An experience system 10 in FIG. 1 includes an acquisition device 11, a providing device 12, and a smartphone 13. The experience system 10 provides, using the providing device 12, stimuli to for experiencer, who is a user of the providing device 12, on the basis of information regarding a recalled person, who evokes a sense of connection in the experiencer, acquired by the acquisition device 11.

Specifically, the acquisition device 11 acquires recalled person information, which is time-series information regarding the recalled person. Examples of the recalled person information include biological information regarding the recalled person, such as a time-series pulse wave, a heartbeat, an electroencephalogram, perspiration, a body temperature, an odor, and a skin potential, audio or text data regarding a message addressed to the experiencer input by the recalled person, a moving image of the recalled person, and the like.

The recalled person may perform the input work of the recalled person information using the acquisition device 11 alone or together with the experiencer. In a case where the recalled person performs the input work together with the experiencer, the input work functions as symbol grounding, and the experiencer can more feel stimuli provided by the providing device 12 as the recalled person. If the experiencer has experience of performing this input work, even in a case where the recalled person performs the input work alone, the input work easily functions as the symbol grounding when the providing device 12 provides the stimuli. On the other hand, in a case where the experiencer has not experienced the input work, the providing device 12 can give the experiencer a fresh impression by providing the stimuli for the experiencer.

The acquisition device 11 records recalled person information. The acquisition device 11 transmits the recorded recalled person information to the providing device 12. The acquisition device 11 receives gratitude information, such as a message to the recalled person or a gratitude point input by the experiencer, transmitted from the providing device 12. The acquisition device 11 displays the message from the experiencer or the gratitude point to the recalled person on the basis of the gratitude information.

The acquisition device 11 receives an experiencer information table in which the recalled person information transmitted from the providing device 12 is associated with a state estimation index indicating degrees of various states of the experiencer after stimuli are provided on the basis of the recalled person information. The acquisition device 11 displays the experiencer information table to the recalled person as necessary.

The providing device 12 (information processing device) includes a database, and accumulates the recalled person information transmitted from the acquisition device 11 in the database. Note that this database may exist on a cloud, and the providing device 12 may acquire the recalled person information from the acquisition device 11 via the database.

The providing device 12 provides stimuli to the experiencer in a mindfulness and relaxation mode, a connection evoking mode, and a self-compassion mode.

The mindfulness and relaxation mode is an operation mode for bringing the experiencer into a mindful state and a relaxed state. The connection evoking mode is an operation mode that evokes a sense of connection with the recalled person in the experiencer. The self-compassion mode is an operation mode for promoting improvement of self-compassion of the experiencer. Self-compassion means compassion for oneself, and is a psychological state or a psychological technique of valuing oneself so as to be kind to others. With self-compassion, a person can keep a positive feeling even in a stressful situation.

The provision of the stimuli in the mindfulness and relaxation mode is initiated prior to the provision of the stimuli in the connection evoking mode. The provision of the stimuli in the self-compassion mode is initiated prior to the provision of the stimuli in the connection evoking mode.

In a case where the operation mode is the mindfulness and relaxation mode, the providing device 12 provides stimuli that activate the parasympathetic nerve system or leads to mindfulness as stimuli that bring the experiencer into a relaxed state. In addition, the providing device 12 also provides stimuli for promoting secretion of oxytocin. Specifically, the providing device 12 provides guidance to a mindful state by a voice guidance message of mediation or vibration mediation, a relaxation effect by a video (moving image) of relaxation music, healing music, natural sound, natural scenery, burning fire, or the like, and an effect of promoting secretion of oxytocin by a scent of lavender or the like, massage, a comfortable temperature, or the like.

Note that oxytocin is an endocrine hormone related to trust, bond, and affection, and is considered to be an important hormone that promotes a sense of connection with an attached person.

The providing device 12 also provides stimuli that cultivates a sense of being in the “here and now” as stimuli for bringing the experiencer into the mindful state. Specifically, the providing device 12 provides a voice guidance message, vibration, or the like of meditation such as respiration mediation that focuses on breathing. The experiencer can be in the mindful state by, for example, meditating to the voice guidance message or meditating while concentrating on vibration. Note that the mindfulness and relaxation mode may be a mode for bringing the experiencer into only the mindful state.

In a case where the operation mode is the connection evoking mode, the providing device 12 provides stimuli for evoking a sense of connection with the recalled person in the experiencer on the basis of the recalled person information. The connection evoking mode includes a passive mode in which stimuli are provided regardless of an input from the experiencer and an active mode in which stimuli are provided according to an input from the experiencer.

In the passive mode, the experiencer unilaterally enjoys the stimuli, but in the active mode, the experiencer enjoys the stimuli by performing an act of expressing his/her feeling in return toward the recalled person. The act of expression a feeling in return toward the recalled person is, for example, an act of gripping or touching back while thinking of the recalled person, an act of returning a message to the recalled person, or the like.

The stimuli in the connection evoking mode include physical stimuli such as a vibration corresponding to the heartbeat of the recalled person and a comfortable pressure (touch) to the skin (C fiber) reproducing a feel of a hand and cognitive stimuli such as a message from the recalled person to the experiencer. Oxytocin is secreted by the physical stimuli and the cognitive stimuli.

The secretion of oxytocin by physical stimuli (in particular skin (C fiber) stimuli) is described, for example, in Francis McGlone 1, Johan Wessberg 2, Hakan Olausson “Discriminative and affective touch: sensing and feeling” Neuron, 2014 May 21.

An effect of cognitive stimuli (→top-down effect; recognition of who is touching is important) on the skin (C fiber) is described, for example, in Chantal Triscoli, Hakan Olausson, Uta Sailer, Hanna Ignell, Ilona Croy, “CT-optimized skin stroking delivered by hand or robot is comparable” Front Behav Neurosci, 7:208, 2013.

The effect of cognitive stimuli (→priming effect) on skin (C fiber) stimuli is also described in Ellingsen, Dan-Mikael Wessberg, Johan Chelnokova, Olga Olausson, Hakan Laeng, Bruno Leknes, Siri, “In touch with your emotions: Oxytocin and touch change social impressions while others' facial expressions can alter touch.” psychneuroendocrinology, 39, 11-20, 2014.

In a case where the operation mode is the self-compassion mode, the providing device 12 provides stimuli for promoting improvement of self-compassion of the experiencer. Specifically, as described in Non-Patent Document 1, there are three elements as elements for improving the self-compassion of the experiencer. The first element is the mindful state, the second element is gentleness to oneself, and the third element is recognition of common humaneness.

Here, since the experiencer is in the mindful state in the mindfulness and relaxation mode, the providing device 12 provides stimuli that promote at least one of the gentleness to oneself and the recognition of common humaneness in the self-compassion mode.

Examples of the stimuli for promoting the gentleness to oneself include a voice guidance message of compassion meditation in which gentleness to the recalled person cultivated by the sense of connection evoked in the experiencer in the connection evoking mode is sublimated to the gentleness to oneself. Examples of the stimuli for promoting the recognition of common humaneness include a voice message with content of “Humans are not perfect. Imperfection is a common experience of humans”.

The providing device 12 acquires experiencer information including a degree of autonomic nerve activity including a time-series heartbeat, degrees of activity of the sympathetic nerve and the parasympathetic nerve of the experiencer, biological information such as brain waves, voice, images, text data, and the like. The providing device 12 receives a subjective index indicating a state of the experiencer input before and after the provision of the stimuli by the experiencer transmitted from the smartphone 13. The providing device 12 generates a state estimation index as feedback information indicating a change of the experiencer due to the stimuli on the basis of the experiencer information and the subjective index after the provision of the stimuli.

The providing device 12 stores, for each experiencer, an experiencer information table in which stimulus information indicating stimuli provided for the experiencer in each operation mode, a recalled person information ID of recalled person information used for the stimuli, and a state estimation index of the experiencer after the provision of the stimuli are associated with each other in the database.

The providing device 12 determines stimuli to be provided in each operation mode on the basis of the experiencer information table and the like.

For example, the providing device 12 can enhance memories of stimuli provided in the past by selecting the stimuli as the stimuli to be provided this time. The providing device 12 can prevent the experiencer from becoming bored by selecting stimuli that differ from ones previously provided. The providing device 12 performs artificial intelligence (AI) analysis or the like on the experiencer information table to select optimal stimuli according to experiencer information and the like before the provision of the stimuli as the stimuli to be provided this time.

The providing device 12 transmits the state estimation index and advice information indicating a message of advice to the experiencer to the smartphone 13.

The providing device 12 acquires gratitude information input by the experiencer, and transmits the gratitude information to the acquisition device 11. The experiencer may input the gratitude information after the provision of the stimuli, or may input the gratitude information at a time of registering the recalled person information to the database. The providing device 12 transmits the experiencer information table to the acquisition device 11.

The smartphone 13 transmits the subjective index to the providing device 12 in accordance with inputs from the experiencer before and after the provision of stimuli. The subjective index is, for example, a UCLA loneliness scale, a subjective index of a sense of connection, a self-compassion scale, a mindful attention awareness scale (MASS), or the like.

Details of the UCLA loneliness scale are described, for example, in Yuzuri Masuda, Etsuko Tadaka, and Yuka Dai, “Reliability and Validity of the Japanese Version of the UCLA Loneliness Scale Version 3 among the Older Population”, Journal of Japan Academy of Community Health Nursing, published in 2012.

Details of the self-compassion scale are described in, for example, Kohki Arimitsu, Yasuhiko Aoki, Miyuki Kokita, Ayano Tada, and Riko Togashi, “Construction and Validation of a Short Form of the Japanese version of the Self-Compassion Scale”, Komazawa annual reports of psychology, issue 18, published in 2016.

Details of the MASS are described, for example, in Masahiro Fujino, Shogo Kajimura, and Michio Nomura “Development and Validation of the Japanese Version of the Mindful Attention Awareness Scale Using Item Response Theory Analysis”, The Japanese Journal of Personality, vol. 24, issue 1, published in 2015.

The smartphone 13 displays the state estimation index and the advice information transmitted from the providing device 12.

### Configuration Example of Acquisition Device

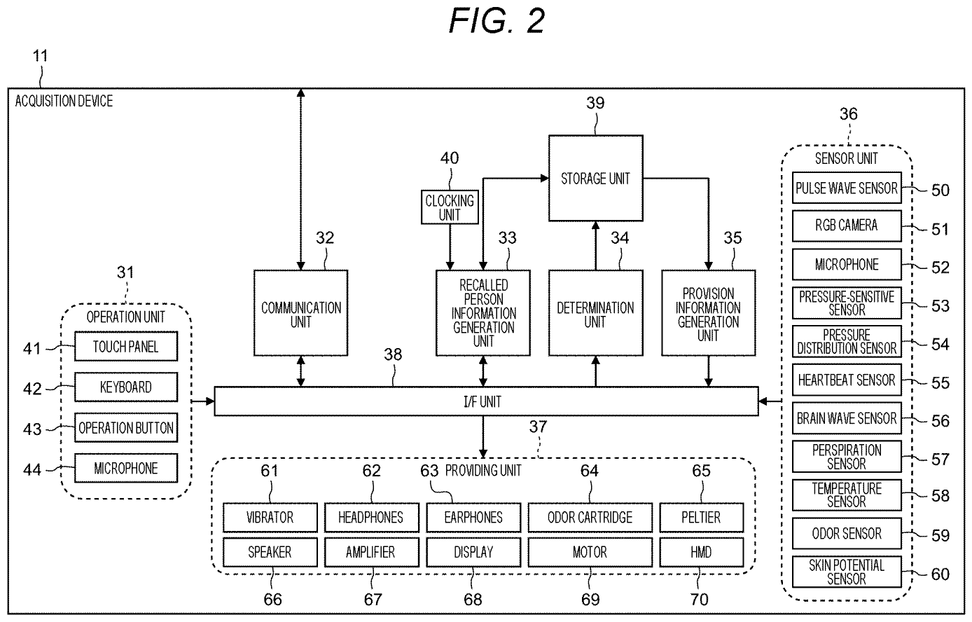

FIG. 2 is a block diagram illustrating a configuration example of the acquisition device 11 in FIG. 1.

The acquisition device 11 of FIG. 2 is configured by connecting an operation unit 31, a communication unit 32, a recalled person information generation unit 33, a determination unit 34, a provision information generation unit 35, a sensor unit 36, and a providing unit 37 via an interface (IF) unit 38. A storage unit 39 and a clocking unit 40 are connected to the recalled person information generation unit 33, and the storage unit 39 is connected to the determination unit 34 and the provision information generation unit 35.

The operation unit 31 includes a touch panel 41, a keyboard 42, an operation button 43, and a microphone 44. The touch panel 41, the keyboard 42, and the operation button 43 receive an operation from the recalled person and supply an operation signal indicating the operation to the IF unit 38.

For example, the recalled person inputs predetermined characters by operating the touch panel 41, the keyboard 42, or the operation button 43, and the IF unit 38 generates text data on the basis of an operation signal indicating the operation. The microphone 44 acquires a voice related to an operation from the recalled person and supplies an operation signal corresponding to the voice to the IF unit 38.

The communication unit 32 communicates with the providing device 12 via a network or the like. For example, the communication unit 32 transmits the recalled person information supplied from the recalled person information generation unit 33 via the IF unit 38 to the providing device 12. The communication unit 32 receives the gratitude information and the experiencer information table transmitted from the providing device 12, and supplies the gratitude information and the experiencer information table to the IF unit 38.

The recalled person information generation unit 33 generates recalled person information on the basis of the text data and the voice supplied from the operation unit 31 through the IF unit 38 and the sensor information supplied from the sensor unit 36 through the IF unit 38. At this time, the recalled person information generation unit 33 adds a recalled person ID, which is an ID of the recalled person, recalled person information ID, which is an ID of the recalled person information, and the like to the recalled person information.

The recalled person information generation unit 33 supplies the recalled person information to the storage unit 39 together with the time information indicating the current time and the like supplied from the clocking unit 40 and stores the recalled person information. The recalled person information generation unit 33 reads the recalled person information stored in the storage unit 39 and supplies the recalled person information to the communication unit 32 via the IF unit 38.

The determination unit 34 selects, as provision data, data to be provided for the recalled person information from the gratitude information, the experiencer information table, and the like supplied from the communication unit 32 via the IF unit 38. The determination unit 34 supplies and stores the provision data to and in the storage unit 39.

The provision information generation unit 35 reads the provision data stored in the storage unit 39 and generates provision information for controlling the providing unit 37 in such a way as to provide the provision data to the recalled person. The provision information generation unit 35 supplies the provision information to the providing unit 37 via the IF unit 38.

The sensor unit 36 includes a pulse wave sensor 50, an RGB camera 51, a microphone 52, a pressure-sensitive sensor 53, a pressure distribution sensor 54, a heartbeat sensor 55, a brain wave sensor 56, a perspiration sensor 57, a temperature sensor 58, an odor sensor 59, and a skin potential sensor 60. At least a part of the sensor unit 36 functions as a measurement unit that measures biological information.

The pulse wave sensor 50 measures a pulse wave of the recalled person and acquires information indicating the pulse wave as sensor information. The RGB camera 51 photographs the recalled person and acquires an RGB image of the recalled person as sensor information. The microphone 52 acquires surrounding sound such as a message to the experiencer from the recalled person as sensor information.

The pressure-sensitive sensor 53 measures pressure of pressing force at a time when the recalled person performs an action such as grasping, and acquires information indicating the pressure as sensor information. The pressure distribution sensor 54 measures pressure distribution of pressing force at a time when the recalled person performs an action such as grasping, and acquires information indicating the pressure distribution as sensor information. The heartbeat sensor 55 measures a heartbeat and a heart rate [bpm] of the recalled person and acquires information indicating the heartbeat and the heart rate as sensor information.

The brain wave sensor 56 measures a brain wave of the recalled person and acquires information indicating the brain wave as sensor information. The perspiration sensor 57 measures perspiration of the recalled person and acquires information indicating the perspiration as sensor information. The temperature sensor 58 measures a body temperature of the recalled person and acquires information indicating the body temperature as sensor information. The odor sensor 59 measures an odor around the recalled person and acquires information indicating the odor and information indicating a type of perfume corresponding to the odor as sensor information.

The skin potential sensor 60 measures a skin potential indicating a tension level of the recalled person and acquires information indicating the skin potential as sensor information. The sensor unit 36 supplies the acquired sensor information to the recalled person information generation unit 33 via the IF unit 38.

The providing unit 37 includes a vibrator 61, headphones 62, earphones 63, an odor cartridge 64, a Peltier 65, a speaker 66, an amplifier 67, a display 68, a motor 69, and a head mounted display (HMD) 70.

The vibrator 61 provides vibration for the recalled person on the basis of the provision information supplied from the provision information generation unit 35 via the IF unit 38. The headphones 62 and the earphones 63 provide a voice for the recalled person on the basis of the provision information. The odor cartridge 64 provides an odor for the recalled person on the basis of the provision information. The Peltier 65 provides a temperature for the recalled person on the basis of the provision information. The Peltier 65 may be a material other than the Peltier as long as the material can provide thermal cooling sensation.

The speaker 66 provides a sound for the recalled person on the basis of the provision information. The amplifier 67 amplifies the sound provided for the recalled person from the headphones 62, the earphones 63, or the speaker 66 on the basis of the provision information. The display 68 provides the recalled person with a still image, a moving image, an image representing a message or sensor information, or the like on the basis of the provision information. The motor 69 drives various mechanisms (not illustrated) on the basis of the provision information, and provides various types of pressure (tactile sense) and humidity for the recalled person. Instead of the motor 69, another actuator may be provided. The HMD 70 provides an image for the recalled person on the basis of the provision information.

The storage unit 39 stores the recalled person information supplied from the recalled person information generation unit 33 and the provision information supplied from the determination unit 34. The clocking unit 40 clocks the current time and supplies the time information for the recalled person information generation unit 33.

Note that, in the acquisition device 11, the microphone 44 and the microphone 52 may be integrated together. At least some of the microphone 44, the microphone 52, the headphones 62, the earphones 63, and the speaker 66 may be integrated together. The display 68 and the HMD 70 may be integrated together. The vibrator 61, the odor cartridge 64, the Peltier 65, the motor 69, the HMD 70, and the like need not be provided.

### Configuration Example of Providing Device

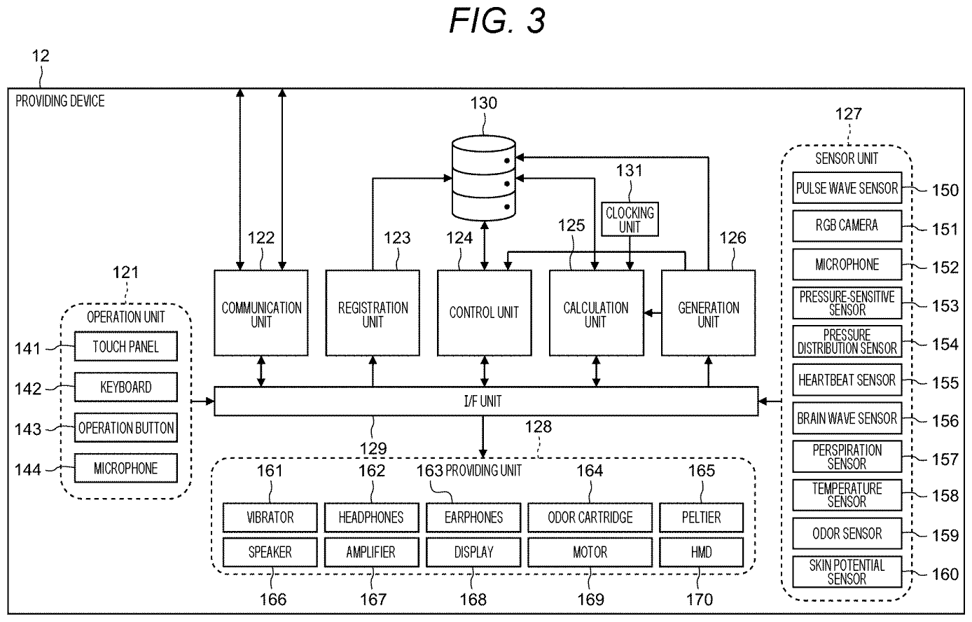

FIG. 3 is a block diagram illustrating a configuration example of the providing device 12 in FIG. 1.

In the providing device 12 of FIG. 3, an operation unit 121, a communication unit 122, a registration unit 123, a control unit 124, a calculation unit 125, a generation unit 126, a sensor unit 127, and a providing unit 128 are connected via an IF unit 129. A database 130 is also connected to the registration unit 123, the control unit 124, the calculation unit 125, and the generation unit 126. A clocking unit 131 is further connected to the calculation unit 125.

The operation unit 121 includes a touch panel 141, a keyboard 142, an operation button 143, and a microphone 144. The operation unit 121 is different from the operation unit 31 in that not the recalled person but the experiencer performs an operation, and because other configurations are similar to those of the operation unit 31, description thereof is omitted.

For example, by operating the operation unit 121, the experiencer inputs an experiencer ID, which is an ID of the experiencer, a recalled person ID of a desired recalled person, and the like, and the IF unit 129 generates the experiencer ID and the recalled person information ID on the basis of an operation signal indicating the operation. The experiencer inputs gratitude information by operating the operation unit 121, and the IF unit 129 generates the gratitude information on the basis of an operation signal indicating the operation.

The communication unit 122 communicates with the acquisition device 11 via a network or the like. For example, the communication unit 122 transmits the gratitude information supplied from the IF unit 129 to the acquisition device 11. The communication unit 122 transmits the experiencer information table supplied from the calculation unit 125 via the IF unit 129 to the acquisition device 11. The communication unit 122 receives recalled person information transmitted from the acquisition device 11 and supplies the recalled person information to the registration unit 123 via the IF unit 129.

The communication unit 122 communicates with the smartphone 13 via a network or the like. For example, the communication unit 122 transmits a state estimation index and advice information supplied from the calculation unit 125 via the IF unit 129 to the smartphone 13. The communication unit 122 receives a subjective index transmitted from the smartphone 13 and supplies the subjective index to the calculation unit 125 via the IF unit 129.

The registration unit 123 registers the recalled person information supplied from the communication unit 122 via the IF unit 129 to a recalled person information table, which is a table of recalled person information stored in the database 130.

The control unit 124 reads, on the basis of a recalled person ID and an experiencer ID supplied from the IF unit 129, a state estimation index, stimulus information, and a recalled person information ID corresponding to the recalled person ID and the experiencer ID from the experiencer information table stored in the database 130. The control unit 124 determines stimuli to be provided for the experiencer in each operation mode on the basis of the state estimation index, the stimulus information, and the recalled person information ID. The control unit 124 supplies the database 130 with stimulus information indicating the determined stimuli in each of the operation modes, the recalled person information ID of the recalled person information used for the stimuli, and the experiencer ID.

The control unit 124 reads, on the basis of the recalled person ID, recalled person information corresponding to the recalled person information ID from the recalled person information table stored in the database 130. The control unit 124 generates stimulus control information for controlling the providing unit 128 in such a way as to provide the stimuli in each operation mode on the basis of the recalled person information, and supplies the stimulus control information to the providing unit 128 via the IF unit 129.

The control unit 124 changes the stimuli in the active mode on the basis of the experiencer information supplied from the generation unit 126. Specifically, the control unit 124 generates stimulus control information for controlling the providing unit 128 in such a way as to provide the stimuli in the active mode after the change, and supplies the stimulus control information to the providing unit 128 via the IF unit 129.

The calculation unit 125 acquires a subjective index supplied from the communication unit 122 via the IF unit 129 and experiencer information supplied from the generation unit 126. The calculation unit 125 (generation unit) calculates a state estimation index on the basis of the subjective index and the experiencer information.

For example, a calculation unit 125 calculates the state estimation index of the evoking of the sense of connection on the basis of a degree of synchronization of the heartbeat or the electroencephalogram in the recalled person information and the experiencer information used for the provided stimuli or a degree of activity of the parasympathetic nerve in the experiencer information. The calculation unit 125 registers the state estimation index together with the time information supplied from the clocking unit 131 to the experiencer information table stored in the database 130.

The calculation unit 125 generates advice information on the basis of the state estimation index. The calculation unit 125 supplies the state estimation index and the advice information to the communication unit 122 via the IF unit 129. The calculation unit 125 reads the experiencer information table from the database 130, and supplies the experiencer information table to the communication unit 122 via the IF unit 129.

The generation unit 126 generates experiencer information on the basis of the sensor information supplied from the sensor unit 127 via the IF unit 129, and supplies the experiencer information to the control unit 124, the calculation unit 125, and the database 130.

A sensor unit 127 includes a pulse wave sensor 150, an RGB camera 151, a microphone 152, a pressure-sensitive sensor 153, a pressure distribution sensor 154, and a heartbeat sensor 155. The sensor unit 127 also includes a brain wave sensor 156, a perspiration sensor 157, a temperature sensor 158, an odor sensor 159, and a skin potential sensor 160.

The sensor unit 127 is different from the sensor unit 36 in that the target for acquiring the sensor information is not the recalled person but the experiencer and the sensor information is supplied to the generation unit 126, and because other points are similar to those of the sensor unit 36, description thereof is omitted.

The providing unit 128 includes a vibrator 161, headphones 162, earphones 163, an odor cartridge 164, a Peltier 165, a speaker 166, an amplifier 167, a display 168, a motor 169, and an HMD 170.

The providing unit 128 is different from the providing unit 37 in that the providing target is not the recalled person but the experiencer and that the provision is performed on the basis of the stimulus control information supplied from the control unit 124, and because other configurations are similar to those of the providing unit 37, description thereof is omitted.

The database 130 stores a recalled person information table and an experiencer information table. The database 130 registers recalled person information supplied from the registration unit 123 to the recalled person information table. The database 130 registers an experiencer ID, a recalled person ID, and stimulus information supplied from the control unit 124 to the experiencer information table. The database 130 registers a state estimation index and time information supplied from the calculation unit 125 to the experiencer information table. The database 130 registers experiencer information supplied from the generation unit 126 to the experiencer information table.

The clocking unit 131 generates time information and supplies the time information to the calculation unit 125.

Note that, in the providing device 12, the microphone 144 and the microphone 152 may be integrated together. At least some of the microphone 144, the microphone 152, the headphones 162, the earphones 163, and the speaker 166 may be integrated together. The display 168 and the HMD 170 may be integrated together.

In the following description, a case where sound is provided by the headphones 162 will be described, but it applies to a case where sound is provided by the earphones 163 or the speaker 166 in a similar manner. In the following description, a case where a moving image or a still image is provided by the display 168 will be described, but it applies to a case where a moving image or a still image is provided by the HMD 170 in a similar manner.

### Configuration Example of Recalled Person Information Table

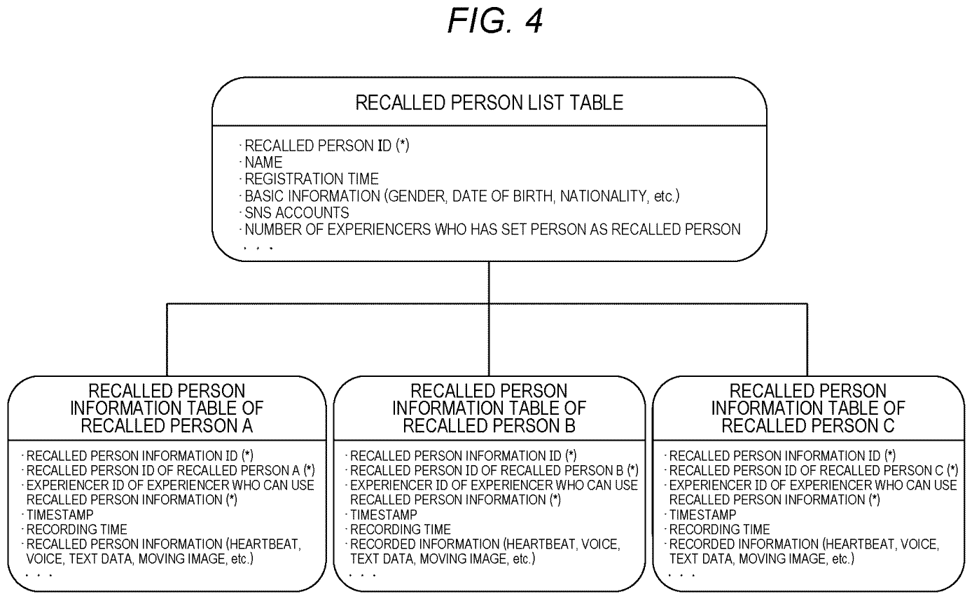

FIG. 4 is a diagram illustrating a configuration example of the recalled person information table stored in the database 130 in FIG. 3.

As illustrated in FIG. 4, the recalled person information table is hierarchically stored in the database 130. Specifically, the database 130 stores a recalled person list table as an upper layer of the recalled person information table. The recalled person list table is a table in which personal information regarding each recalled person is registered in association with a recalled person ID of the recalled person. The personal information includes basic information such as a name, a registration time of the personal information, gender, a date of birth, and nationality, SNS accounts, and the number of experiencers who have set the person as a recalled person.

A recalled person table of each recalled person is stored below the recalled person list table for the recalled person. In the example of FIG. 4, recalled persons who have performed the input work of recalled person information are three recalled persons A to C. In this case, recalled person tables of the recalled persons A to C are stored below the recalled person list table.

In the recalled person table of the recalled person A, a recalled person ID of the recalled person A and an experiencer ID of an experiencer who can use the recalled person information are registered in association with a recalled person information ID of each of pieces of recalled person information regarding the recalled person A. In the recalled person information ID, a timestamp, which is time information at a time when the recalled person information corresponding to the recalled person information ID was recorded (generated), a recording time (acquisition time) of the recalled person information, and the recalled person information are registered. This recording time is calculated, for example, on the basis of times clocked by the clocking unit 40 at a start and an end of generation of the recalled person information.

The recalled person tables of the recalled person B and the recalled person C are also configured similarly to the recalled person table of the recalled person A except that the recalled person A is replaced with the recalled persons B and C, respectively, and description thereof is omitted.

### Example of Information Registered in Recalled Person Table

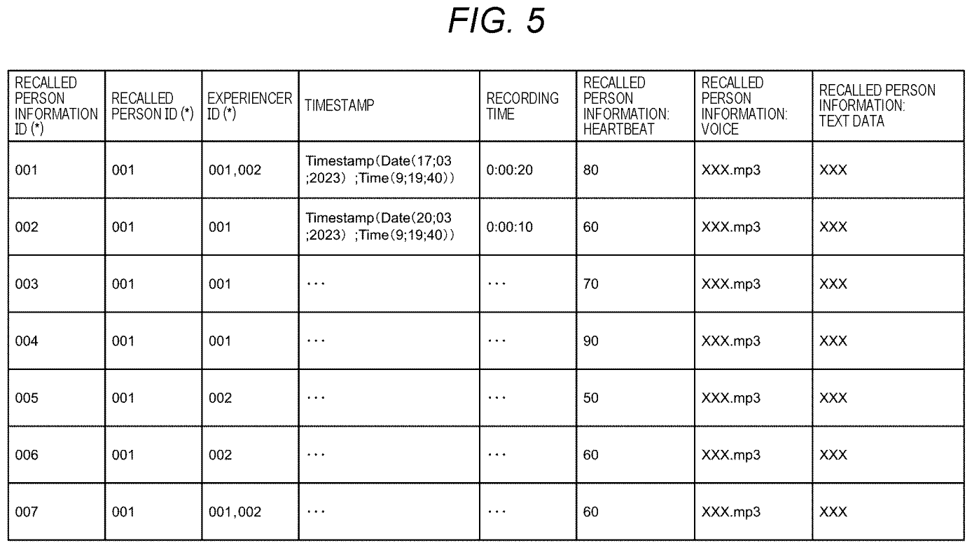

FIG. 5 is a diagram illustrating an example of information registered in the recalled person table of the recalled person A in FIG. 4.

In the example of FIG. 5, a heartbeat, a voice, and text data are generated seven times as the recalled person information regarding the recalled person A. Therefore, the recalled person information table of FIG. 5 includes seven entries. In the example of FIG. 5, for example, recalled person information IDs from “001” to “0007” are added to the seven pieces of recalled person information in order of generation, and each of “001” to “0007” is registered as the recalled person information ID of each entry.

In the example of FIG. 5, the recalled person ID of the recalled person A is “001”, and “001” is registered as the recalled person ID of each entry. In the example of FIG. 5, “001” and “002” are registered as experiencer IDs in the entry of the recalled person information ID “001”. In this case, the recalled person information of the recalled person information ID “001” can be used to provide stimuli for an experiencer with the experiencer ID “001” or “002”.

In the example of FIG. 5, the recalled person information of the recalled person information ID “001” was generated at 9:19:40 on Mar. 17, 2023. Therefore, in the entry of the recalled person information ID “001”, “Timestamp (Date (17;03;2023); Time (9;19;40)) is registered as a timestamp.

In the example of FIG. 5, the recording time of the recalled person information of the recalled person information ID “001” is 20 seconds, and in the entry of the recalled person information ID “001”, “0;00:20”.

In the example of FIG. 5, the heartbeat in recalled person information of a recalled person information ID “001” is 80, a file name of the voice is “xxx.mp3”, and the text data is “xxx”. Therefore, in an entry of the recalled person information ID “001”, 80 is registered as the heartbeat of the recalled person information, “xxx.mp3” is registered as the voice, and “xxx” is registered as the text data.

Note that although description is omitted, an experiencer ID, a timestamp, a recording time, a heartbeat, a voice, and text data are similarly registered in each of entries of recalled person information IDs “002” to “0007”.

Information with “*” at an end in FIGS. 4 and 5 is information that can be referred to from other tables. Specifically, the recalled person IDs of the recalled person list table, and the recalled person information IDs, the recalled person IDs, and the experiencer IDs of the recalled person information tables are information that can be referred to from other tables.

A type of recalled person information registered in the recalled person information tables is set in advance by an owner of the experience system 10.

### External Configuration Example of Part of Providing Device

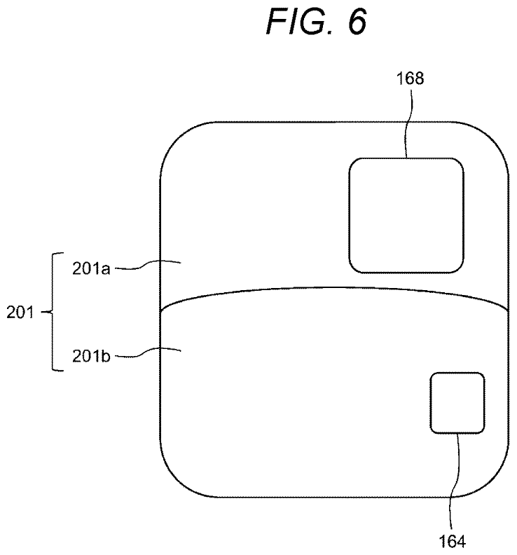

FIG. 6 is a top view illustrating an external configuration example of a part of the providing device 12.

As illustrated in FIG. 6, a mechanism that is driven by a motor 169 of the providing device 12 and that provides various types of pressure for the experiencer includes a pressure providing mechanism 201 of a glove type. The pressure providing mechanism 201 includes an upper mechanism 201a and a lower mechanism 201b covered with a cushion. The lower mechanism 201b is larger than the upper mechanism 201a, and the upper mechanism 201a and the lower mechanism 201b are partially bonded.

The experiencer inserts his/her hand between the upper mechanism 201a and the lower mechanism 201b. When driven by the motor 169, the upper mechanism 201a and the lower mechanism 201b expand and contract pneumatically, thereby providing pressure to the hand of the experiencer between the upper mechanism 201a and the lower mechanism 201b.

The display 168 is provided on an upper surface of the upper mechanism 201a, and the odor cartridge 164 is provided on an upper surface of the lower mechanism 201b.

The pressure providing mechanism 201 may be integrated with the communication unit 122, the registration unit 123, the control unit 124, the calculation unit 125, the generation unit 126, the IF unit 129, the database 130, the clocking unit 131, and the like. The pressure providing mechanism 201 can also be integrated with the vibrator 161, the Peltier 165, the speaker 166, the amplifier 167, the motor 169, and the like. The pressure providing mechanism 201 may also be integrated with the sensor unit 127 other than a brain wave sensor 156. Note that, in the following description, it is assumed that the pressure providing mechanism 201 is integrated with these components.

### Configuration Example of Control Unit

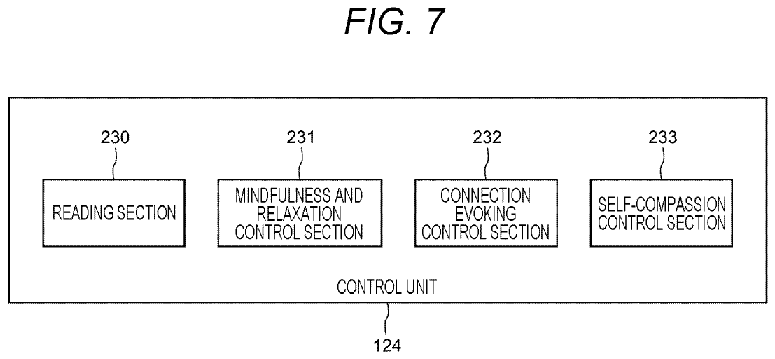

FIG. 7 is a block diagram illustrating a configuration example of the control unit 124 in FIG. 3.

The control unit 124 in FIG. 7 includes a reading section 230, a mindfulness and relaxation control section 231, a connection evoking control section 232, and a self-compassion control section 233.

The reading section 230 reads, on the basis of a recalled person ID and an experiencer ID supplied from the IF unit 129, a state estimation index, stimulus information, and a recalled person information ID corresponding to the recalled person ID and the experiencer ID from the experiencer information table stored in the database 130.

The reading section 230 supplies the read state estimation index and stimulus information in the mindfulness and relaxation mode to the mindfulness and relaxation control section 231. The reading section 230 supplies the read state estimation index, stimulus information in the connection evoking mode, and the recalled person information ID to the connection evoking control section 232. The reading section 230 supplies the read state estimation index and stimulus information in the self-compassion mode to the self-compassion control section 233.

On the basis of the state estimation index supplied from the reading section 230 and the stimulus information in the mindfulness and relaxation mode, the mindfulness and relaxation control section 231 determines stimuli to be provided for the experiencer in the mindfulness and relaxation mode. The mindfulness and relaxation control section 231 supplies stimulus information indicating the stimuli and the experiencer ID to the database 130. The mindfulness and relaxation control section 231 generates stimulus control information for controlling the providing unit 128 in such a way as to provide the stimuli, and supplies the stimulus control information to the providing unit 128 via the IF unit 129.

The connection evoking control section 232 determines stimuli to be provided for the experiencer in the connection evoking mode on the basis of the state estimation index supplied from the reading section 230, the stimulus information in the connection evoking mode, the recalled person information ID, and the experiencer information supplied from the generation unit 126. The connection evoking control section 232 supplies the database 130 with stimulus information indicating the stimuli, the recalled person information ID of the recalled person information used for the stimuli, and the experiencer ID.

The connection evoking control section 232 reads, on the basis of the recalled person ID, recalled person information corresponding to the recalled person information ID from the recalled person information table stored in the database 130. The connection evoking control section 232 generates stimulus control information for controlling the providing unit 128 in such a way as to provide the stimuli in the connection evoking mode on the basis of the recalled person information, and supplies the stimulus control information to the providing unit 128 via the IF unit 129.

The self-compassion control section 233 determines stimuli to be provided for the experiencer in the self-compassion mode on the basis of the state estimation index supplied from the reading section 230 and the stimulus information in the self-compassion mode. The self-compassion control section 233 supplies stimulus information indicating the stimuli and the experiencer ID to the database 130. The self-compassion control section 233 generates stimulus control information for controlling the providing unit 128 in such a way as to provide the stimuli for the user, and supplies the stimulus control information to the providing unit 128 via the IF unit 129.

### Description of Order of Operation Modes

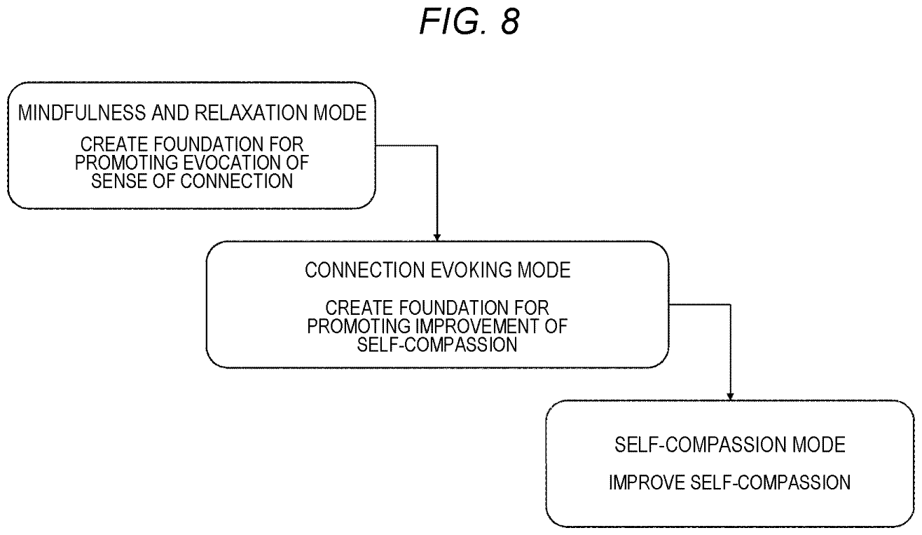

FIG. 8 is a diagram illustrating order of the operation modes by the control unit 124 in FIG. 7.

As illustrated in FIG. 8, first, the mindfulness and relaxation control section 231 starts control such that the providing unit 128 provides the user with the stimuli in the mindfulness and relaxation mode. As a result, the experiencer can relax the mind and body and relax the tension at a start of an experience. In addition, the experiencer enters the mindful state, and can create a foundation for promoting evocation of the sense of connection. That is, the experiencer can feel the sense of connection more easily.

Specifically, the mindful state is a condition in which one transitions from a state in which various emotions and thoughts are racing through the mind or mind-wandering to embodying non-judgmental awareness of the present moment (a sense of here and now). Therefore, the provision of stimuli in the mindfulness and relaxation mode plays a role of grounding to concentrate on evocation of the sense of connection in the connection evoking mode. Mind-wandering is a phenomenon in which attention drifts away from a task at hand and becomes caught in repetitive loops of unrelated thoughts.

After a start of the control by the mindfulness and relaxation control section 231, the connection evoking control section 232 starts the control such that the providing unit 128 provides the user with the stimuli in the connection evoking mode. As a result, the experiencer can recall the sense of connection with the recalled person.

In addition, the experiencer evokes the sense of connection with the recalled person, thereby evoking gentleness (compassion) to the recalled person, and it is easy to direct the gentleness to himself/herself. Physiologically, since oxytocin of the experiencer is enhanced by evoking the sense of connection with the recalled person, the experiencer is more likely to have gentleness to the recalled person and himself/herself. As described above, the experiencer can more easily feel the gentleness to himself/herself, which is an element for improving the self-compassion. That is, the experiencer can create a foundation for promoting improvement of self-compassion.

After a start of the control by the connection evoking control section 232, the self-compassion control section 233 starts the control such that the providing unit 128 provides the user with the stimuli in the self-compassion mode. As a result, the experiencer can improve self-compassion.

Specifically, the experiencer is in the mindful state, which is one of three elements for improving self-compassion, as a result of the stimuli in the mindfulness and relaxation mode. In addition, the experiencer is in a state in which the gentleness to the recalled person is easily sublimated to the gentleness to himself/herself as a result of the stimuli in the connection evoking mode. Then, as a result of the stimuli in the self-compassion mode, the experiencer sublimates the gentleness to the recalled person to the gentleness to himself/herself, and enters a state of being able to feel the gentleness to himself/herself, which is another factor of improving self-compassion. The experiencer also enters a state of being able to recognize common human nature, which is the last one of the factors of improving self-compassion, as a result of the stimuli in the self-compassion mode. Therefore, the experiencer can improve self-compassion.

As described above, since the stimuli in the mindfulness and relaxation mode are provided before the stimuli in the connection evoking mode, it is possible to effectively evoke a sense of connection in the experiencer as compared with a case where the stimuli in the mindfulness and relaxation mode are not provided.

In addition, since the stimuli in the connection evoking mode and the mindfulness and relaxation mode are provided before the stimuli in the self-compassion mode, self-compassion can be improved as compared with a case where only the stimuli in the self-compassion mode are provided.

### Description of Oxytocin Enhancement Loop

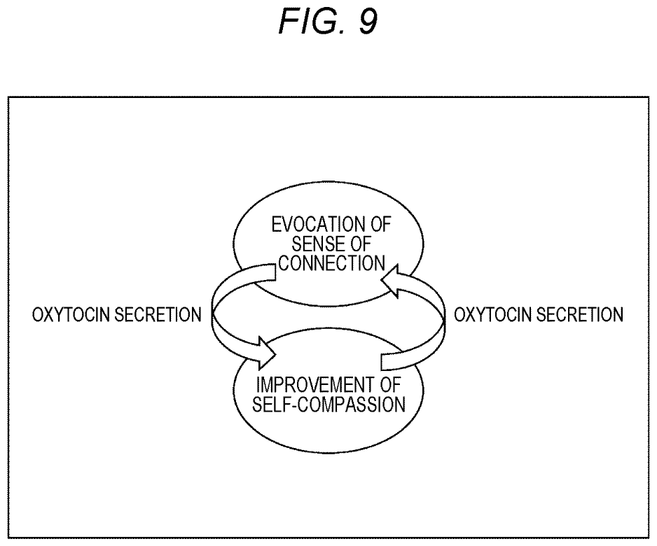

FIG. 9 is a diagram illustrating an enhancement loop for enhancing the secretion of oxytocin of the experiencer formed by providing stimuli using the providing device 12.

As illustrated in FIG. 9, when the stimuli in the connection evoking mode are provided by the providing device 12, the experiencer evokes a sense of connection. As a result, oxytocin is physiologically secreted. Thereafter, when the stimuli in the self-compassion mode are provided, the experiencer improves self-compassion. This may result in further secretion of oxytocin.

Here, as described above, oxytocin promotes evocation of a sense of connection. Therefore, the experiencer can form an oxytocin enhancement loop by continuously using the experience system 10. That is, the improvement of the self-compassion of the experiencer promotes the evocation of the sense of connection at a time of next use of the experience system 10 by the experiencer, which further improves the self-compassion. In addition, this enhancement loop is formed even by one experience of a sense of connection. Oxytocin is secreted by physical stimuli (C fiber of the skin) performed in the experience of the sense of connection, thereby improving a cognitive stimulation effect for thinking about the recalled person (a favorite person, a trusted person, or the like). Furthermore, the evocation of the recalled person itself also promotes oxytocin secretion, which also promotes the oxytocin secretion by the physical stimuli (a synergistic effect of top-down (cognitive stimuli) and bottom-up (physical stimuli)).

Since attachment to the recalled person is formed in the experiencer by the evocation of the sense of connection with the recalled person, the experience system 10 can promote continuous use by the experiencer.

On the other hand, in a case where the experiencer receives a psychoclinical approach under guidance of an expert, such as a mindful self-compassion 8-week program, to improve the self-compassion, a period of the approach is limited. In a case where the experiencer improves self-compassion using a cognitive behavioral therapy application such as a mindfulness application, it is difficult for the experiencer to continue using the cognitive behavioral therapy application because attachment is not formed. Therefore, it is difficult to obtain an effect such as self-transformation, which is a medium- to long-term effect due to improvement of self-compassion, using a psychoclinical approach or a cognitive behavioral therapy application.

### Example of Physical Stimuli in Active Mode

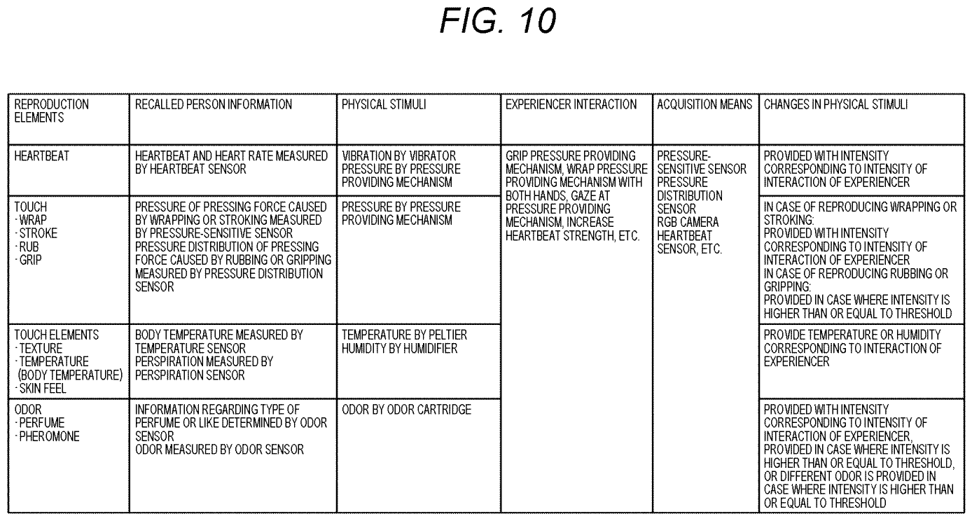

FIG. 10 is a table illustrating an example of physical stimuli in the active mode.

In a first row from a top of the table in FIG. 10, items “reproduction elements”, “recalled person information”, “physical stimuli”, “experiencer interaction, “acquisition means”, and “changes in physical stimuli” are described. In each of second to fifth rows from the top, information corresponding to the corresponding item is described.

The information corresponding to the item “reproduction elements” is an element of the recalled person to be reproduced by providing the physical stimuli on the basis of the recalled person information in the active mode. The information corresponding to the item “recalled person information” is recalled person information to be used to provide the physical stimuli, and the information corresponding to the item “physical stimuli” is the physical stimuli. The information corresponding to the item “experiencer interaction” is an interaction of the experiencer provided with the physical stimuli in the active mode, and the item “acquisition means” is acquisition means for acquiring sensor information corresponding to the interaction. The item “changes in physical stimuli” is changes in the physical stimuli in the active mode corresponding to the interaction.

Specifically, the information corresponding to the item “reproduction elements” and “recalled person information” in the second row from the top of the table of FIG. 10 is “heartbeat” and “heartbeat and heart rate measured by heartbeat sensor”, respectively. The information corresponding to the items “physical stimuli” and “experiencer interaction” is “vibration by vibrator, pressure by pressure providing mechanism” and “gripping, wrapping, gazing, and increasing heartbeat strength of pressure providing mechanism etc.”, respectively. The information corresponding to the items “acquisition means” and “changes in physical stimuli” is “pressure-sensitive sensor, pressure distribution sensor, RGB camera, heartbeat sensor, etc.” and “provided with intensity corresponding to interaction of experiencer”, respectively.

That is, in this case, in the active mode, on the basis of the heartbeat and the heart rate measured by the heartbeat sensor 55 in the recalled person information, vibration by the vibrator 161 or pressure by the pressure providing mechanism 201 is provided as a physical stimulus that reproduces the heartbeat of the recalled person.

After the physical stimulus is provided, the connection evoking control section 232 estimates the interaction of the experiencer on the basis of the experiencer information corresponding to the sensor information from the pressure-sensitive sensor 153 and the pressure distribution sensor 154. Specifically, the connection evoking control section 232 estimates that the experiencer is holding the pressure providing mechanism 201 with one hand or wrapping the pressure providing mechanism 201 with both hands as an interaction on the basis of the experiencer information.

The connection evoking control section 232 estimates that the experiencer is gazing at the pressure providing mechanism 201 as an interaction on the basis of the experiencer information corresponding to the sensor information from the RGB camera 151. The connection evoking control section 232 estimates that the heartbeat strength is increasing as an interaction of the experiencer on the basis of the experiencer information corresponding to the sensor information from the heartbeat sensor 155.

In a case where the connection evoking control section 232 estimates that an interaction that makes the experiencer psychologically closer to the recalled person as described above has occurred, strength of the physical stimulus to be provided is changed on the basis of strength of the interaction.

The information corresponding to the item “reproduction elements” in the third row from the top of the table in FIG. 10 is “touch (wrap, stroke, rub, grip)”. The information corresponding to the item “recalled person information” is “pressure distribution of pressure due to wrapping or stroking measured by pressure-sensitive sensor and pressure due to rubbing and gripping measured by pressure distribution sensor”. The information corresponding to the item “physical stimuli” is “pressure by pressure providing mechanism”. The information corresponding to the items “experiencer interaction” and “acquisition means” is similar to the second row from the top. The information corresponding to the item “changes in physical stimuli” is “in case of reproducing wrapping or stroking: provided with intensity corresponding to interaction of experiencer, in caser of reproducing rubbing or gripping: provided in case intensity is higher than or equal to threshold”.

That is, in this case, in the active mode, on the basis of the pressure measured by the pressure-sensitive sensor 53 in the recalled person information, pressure by the pressure providing mechanism 201 is provided as a physical stimulus that reproduces wrapping or stroking of the recalled person. Alternatively, on the basis of the pressure distribution measured by the pressure distribution sensor 54 in the recalled person information, pressure by the pressure providing mechanism 201 is provided as a physical stimulus that reproduces rubbing or gripping of the recalled person.

After this physical stimulus is provided, the connection evoking control section 232 estimates occurrence of an interaction that makes the experiencer psychologically closer to the recalled person, similarly to after the provision of the physical stimulus that reproduces the heartbeat. In the case of estimating that an interaction that makes the experiencer psychologically closer to the recalled person has occurred after a physical stimulus that reproduces wrapping or stroking is provided, the connection evoking control section 232 performs control such that the physical stimulus is provided with an intensity corresponding to intensity of the interaction. In the case of estimating that an interaction that makes the experiencer psychologically closer to the recalled person has occurred after a physical stimulus that reproduces rubbing or gripping is provided, the connection evoking control section 232 performs control such that the physical stimulus is provided in a case where the intensity of the interaction is higher than or equal to a threshold.

The information corresponding to the item “reproduction elements” in the fourth row from the top of the table in FIG. 10 is “touch elements (texture, temperature (body temperature), and skin feel)”. The information corresponding to the item “recalled person information” is “body temperature measured by temperature sensor, perspiration measured by perspiration sensor”. The information corresponding to the item “physical stimuli” is “temperature by Peltier, humidity by humidifier”. The information corresponding to the items “experiencer interaction” and “acquisition means” is similar to the first row from the top. The information corresponding to the item “changes in physical stimuli” is “provision of temperature and humidity according to interaction of experiencer”.

That is, in this case, a physical stimulus that reproduces touch elements such as texture, temperature, and skin feel of the recalled person is provided in the active mode. Specifically, the temperature is provided by the Peltier on the basis of the temperature measured by the temperature sensor 58 in the recalled person information, and the humidity is provided by a humidifier (not illustrated) on the basis of the perspiration measured by the perspiration sensor 57. The humidifier is driven by the motor 169.

After this physical stimulus is provided, the connection evoking control section 232 estimates occurrence of an interaction that makes the experiencer psychologically closer to the recalled person, similarly to after the provision of the physical stimulus that reproduces the heartbeat. In the case of estimating that this interaction has occurred, the connection evoking control section 232 performs control such that a temperature and humidity corresponding to the interaction are provided as a physical stimulus. For example, in the case of estimating that this interaction has occurred, the connection evoking control section 232 performs control in such a way as to increase the temperature and humidity to be provided.

Note that in a case where the connection evoking control section 232 has estimated occurrence of an interaction that makes the experiencer psychologically more distant from the recalled person on the basis of the experiencer information and estimates that the interaction has occurred, the connection evoking control section 232 may perform control in such a way as to lower the temperature and humidity to be provided.

The information corresponding to the item “reproduction elements” in the fifth row from the top of the table in FIG. 10 is “odor (perfume, pheromone)”. The information corresponding to the item “recalled person information” is “information such as type of perfume determined by odor sensor, odor measured by odor sensor”. The information corresponding to the item “physical stimuli” is “odor by odor cartridge”. The information corresponding to the items “experiencer interaction” and “acquisition means” is similar to the second row from the top. The information corresponding to the item “changes in physical stimuli” is “provided with intensity corresponding to interaction of experiencer, provided in case intensity is higher than or equal to threshold, or different odor is provided in case where intensity is higher than or equal to threshold”.

That is, in this case, in the active mode, an odor by the odor cartridge 64 is provided as a physical stimulus that reproduces an odor of a perfume or a pheromone of the recalled person on the basis of information regarding a type of perfume determined by the odor sensor 59 in the recalled person information or a measured odor.

After this physical stimulus is provided, the connection evoking control section 232 estimates occurrence of an interaction that makes the experiencer psychologically closer to the recalled person, similarly to after the provision of the physical stimulus that reproduces the heartbeat. In the case of estimating that this interaction has occurred, the connection evoking control section 232 performs control in such a way as to provide an odor with intensity corresponding to the intensity of the interaction. Alternatively, in a case where the intensity of the interaction is higher than or equal to a threshold, the connection evoking control section 232 performs control in such a way as to provide an odor or performs control in such a way as to provide an odor different from a previous odor.

### Example of Cognitive Stimuli in Active Mode

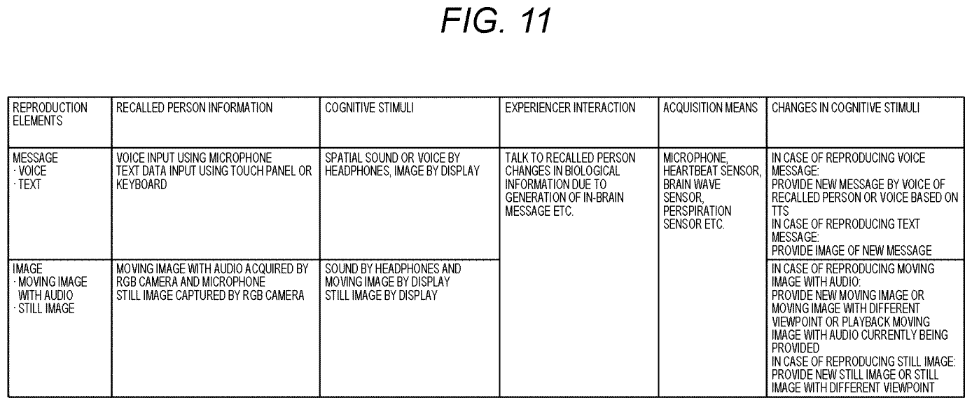

FIG. 11 is a table illustrating an example of cognitive stimuli in the active mode.

In a first row from a top of the table in FIG. 11, items “reproduction elements”, “recalled person information”, “cognitive stimuli”, “experiencer interaction, “acquisition means”, and “changes in cognitive stimuli” are described. In each of second and third rows from the top, information corresponding to the corresponding item is described.

The information corresponding to the item “reproduction elements” is an element of the recalled person to be reproduced by providing the cognitive stimulus on the basis of the recalled person information in the active mode. The information corresponding to the item “recalled person information” is recalled person information to be used to provide the cognitive stimulus, and the information corresponding to the item “cognitive stimuli” is the cognitive stimulus. The information corresponding to the item “experiencer interaction” is an interaction of the experiencer provided with the cognitive stimulus in the active mode, and the item “acquisition means” is acquisition means for acquiring sensor information corresponding to the interaction. The item “changes in cognitive stimuli” is changes in the cognitive stimuli in the active mode corresponding to the interaction.

Specifically, the information corresponding to the item “reproduction elements” in the second row from the top of the table in FIG. 11 is “message (voice, text)”. The information corresponding to the “recalled person information” is “voice acquired by microphone, text data input using touch panel or keyboard”. The information corresponding to the items “cognitive stimuli” and “experiencer interaction” is “spatial sound and voice by headphones, image by display” and “changes in biological information by generation of in-brain message spoken to recalled person etc.”, respectively. The information corresponding to the items “acquisition means” and “changes in cognitive stimuli” is “microphone, heartbeat sensor, brain wave sensor, perspiration sensor, etc.” and “provide new message by voice of recalled person or voice based on TTS, provide image of new message”, respectively.

That is, in this case, in the active mode, a spatial sound or a voice by the headphones 162 is provided as the cognitive stimulus for reproducing a voice message of the recalled person on the basis of a voice input using the microphone 52 in the recalled person information. Alternatively, on the basis of text data input using the touch panel 41 or the keyboard 42 in the recalled person information, an image by the display 168 is provided as a cognitive stimulus for reproducing a text message of the recalled person.

After the cognitive stimulus is provided, the connection evoking control section 232 estimates that the experiencer is talking to the recalled person as an interaction on the basis of the experiencer information corresponding to the sensor information from the microphone 152. The connection evoking control section 232 estimates that the biological information has changed since the experiencer has generated an in-brain message as the interaction on the basis of the experiencer information corresponding to the sensor information from the heartbeat sensor 155, the brain wave sensor 156, the perspiration sensor 157, and the like.

In the case of estimating that an interaction that makes the experiencer psychologically closer to the recalled person has occurred as described above after the cognitive stimulus for reproducing a voice message is provided, the connection evoking control section 232 performs control in such a way as to provide a new message by the voice of the recalled person. Alternatively, the connection evoking control section 232 performs control in such a way as to provide a new message a voice based on text-to-speech (TTS). In the case of estimating that an interaction that makes the experiencer psychologically closer to the recalled person has occurred as described above after the cognitive stimulus for reproducing a text message is provided, the connection evoking control section 232 performs control in such a way as to provide an image of a new message. This new message is selected from, for example, the recalled person information.

The information corresponding to the item “reproduction elements” in the third row from the top of the table in FIG. 11 is “image (moving image with audio, still image)”. The information corresponding to the item “recalled person information” is “moving image with audio acquired by RGB camera and microphone, still image captured by RGB camera”. The information corresponding to the item “cognitive stimuli” is “sound by headphones and moving image by display, still image by display”. The information corresponding to the items “experiencer interaction” and “acquisition means” is similar to the second row from the top. The information corresponding to the item “changes in cognitive stimuli” is “in case of reproducing moving image with audio: provide new moving image or moving image with different viewpoint or playback moving image with audio currently being provided, in case of reproducing still image: provide new still image or still image with different viewpoint”.

That is, in this case, in the active mode, a cognitive stimulus for reproducing a moving image with audio of the recalled person is provided on the basis of a moving image with audio acquired by the RGB camera 51 and the microphone 52 in the recalled person information. Specifically, a voice by the headphones 162 and a moving image by the display 168 are provided as a cognitive stimulus for reproducing the moving image with audio of the recalled person. Alternatively, on the basis of a still image captured by the RGB camera 51 in the evoking target information, a still image by the display 168 is provided as a cognitive stimulus for reproducing the still image of the recalled person.

After this cognitive stimulus is provided, the connection evoking control section 232 estimates occurrence of an interaction that makes the experiencer psychologically closer to the recalled person, similarly to after the provision of the cognitive stimulus that reproduces a voice message or a text message. In the case of estimating that an interaction that makes the experiencer psychologically closer to the recalled person after the provision of the cognitive stimulus that reproduces a moving image with audio, the connection evoking control section 232 performs control in such a way as to provide a new moving image or a moving image with a different viewpoint or playback a currently provided moving image with audio. In the case of estimating that an interaction that makes the experiencer psychologically closer to the recalled person after the provision of the cognitive stimulus that reproduces a still image, the connection evoking control section 232 performs control in such a way as to provide a new still image or a still image with a different viewpoint.

### Example of How Providing Device Provides Stimuli

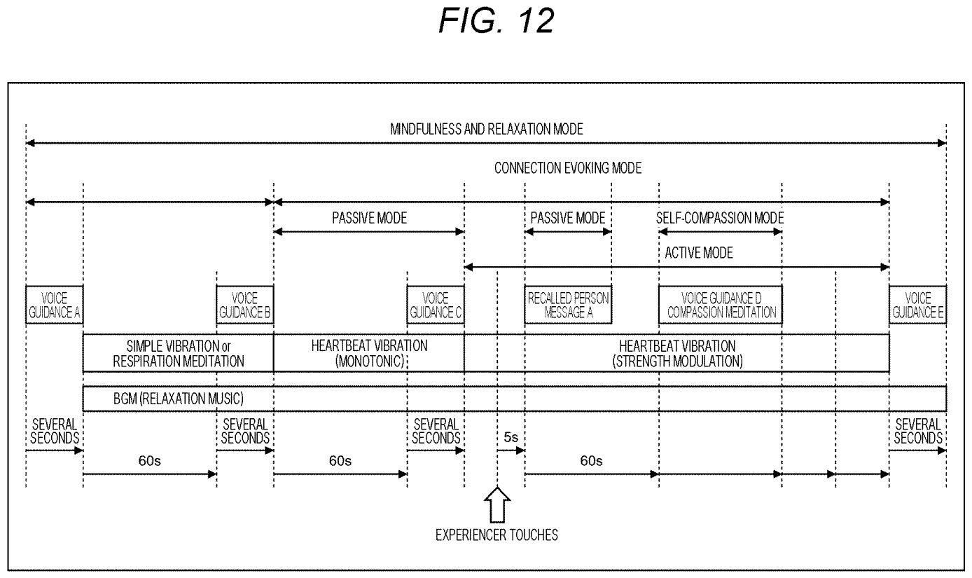

FIG. 12 is a diagram illustrating an example of how the providing device 12 provides stimuli.

In the example of FIG. 12, first, the mindfulness and relaxation control section 231 of the providing device 12 causes the headphones 62 to provide a sound of voice guidance A for several seconds. Voice guidance A includes an experience start message that prompts insertion of both hands into the pressure providing mechanism 201 and an introduction message of stimuli in the mindfulness and relaxation mode.

The experience start message is, for example, “Now you are participating in the experience of feeling connected to oo (the name of the recalled person). Please put both hands into the cushion (pressure providing mechanism 201) in front of you and slowly close your eyes to relax”. The introduction message is, for example, “Slowly breathe, and focus on your own breathing”. The sound of the introduction message is provided, for example, after a predetermined time from an end of the provision of the sound of the experience start message.

After providing the sound of voice guidance A, the mindfulness and relaxation control section 231 causes the vibrator 161 to provide simple vibration and the headphones 162 to provide relaxation music as background music (BGM) as stimuli in the mindfulness and relaxation mode. As a result, the experiencer can concentrate on the vibration while listening to the relaxation music (mindfulness by the vibration). Alternatively, the mindfulness and relaxation control section 231 can also provide respiration mediation by a guidance message instead of simple vibration. In this case, the experiencer can practice mindfulness by concentrating on breathing.

60 seconds after the start of the provision of the stimuli in the mindfulness and relaxation mode, the connection evoking control section 232 causes the headphones 62 to provide a sound of voice guidance B for several seconds. Voice guidance B is, for example, “Now, feel the beating message to you from oo (the name of the recalled person), and remember the person”.

After providing the sound of voice guidance B, the mindfulness and relaxation control section 231 causes the vibrator 161 to stop providing the simple vibration. The connection evoking control section 232 starts to cause the vibrator 161 to provide heartbeat vibration on the basis of the information regarding the heartbeat and the heart rate in the recalled person information as a physical stimulus in the passive mode of the connection evoking mode. As a result, the experiencer can feel the heartbeat of the recalled person.

60 seconds after the start of the provision of the physical stimulus, the connection evoking control section 232 causes the headphones 62 to provide a sound of voice guidance C for several seconds. Voice guidance C is, for example, “Now, let your touch to convey your feeling to the person. Put out one hand and touch the cushion (pressure providing mechanism 201) to feel the heartbeat of the person and receive a new message”.

As a result, the experiencer takes out one of the hands inserted into the pressure providing mechanism 201 and touches the upper mechanism 201a. As a result, the pressure-sensitive sensor 153 provided in the upper mechanism 201a measures the pressure.

After 5 seconds from the measurement of the pressure, the connection evoking control section 232 starts to provide a sound of message A of the recalled person in the recalled person information as a cognitive stimulus in the passive mode of the connection evoking mode on the basis of the voice of message A. As a result, the experiencer can receive the voice message of the recalled person and think about the recalled person.

On the other hand, after providing voice guidance C, the connection evoking control section 232 ends the provision of the physical stimulus in the passive mode. The connection evoking control section 232 then starts to cause the vibrator 161 to provide heartbeat vibration on the basis of the information regarding the heartbeat and the heart rate of the recalled person in the recalled person information and the experiencer information as a physical stimulus in the active mode. As a result, the experiencer can feel the heartbeat vibration according to his/her interaction.

Specifically, after touching the upper mechanism 201a with one hand, the experiencer grips the upper mechanism 201a with the hand or wraps the upper mechanism 201a with both hands while feeling the heartbeat vibration provided by the vibrator 161. As a result, the pressure-sensitive sensor 153 provided in the upper mechanism 201a measures the pressure, and the pressure distribution sensor 154 measures pressure distribution. The connection evoking control section 232 estimates that the experiencer is gripping the pressure providing mechanism 201 with one hand or wrapping the pressure providing mechanism 201 with both hands as an interaction on the basis of the pressure or the pressure distribution. The connection evoking control section 232 then controls the vibrator 161 in such a way as to provide the heartbeat vibration with an intensity corresponding to grip strength or wrapping strength based on the measured pressure or pressure distribution.

The experiencer can enhance the sense of connection to the recalled person by interacting with the pressure providing mechanism 201 while thinking about the recalled person. For example, Hajime Yamaguchi, “[Illustrated Guide] Skin Therapy to Relieve Stress from the Brain”, Seishun Publishing Co., Ltd., published on Jul. 1, 2018, describes a research report in which oxytocin is secreted more in a massaging person than in a massaged person. That is, acting while thinking about others enhances oxytocin secretion. As a result, the oxytocin enhancement loop is further enhanced.

60 seconds after the provision of message A of the recalled person is started, the self-compassion control section 233 causes the headphones 162 to provide sounds of voice guidance D and guidance messages of compassion meditation as stimuli in the self-compassion mode.

Voice guidance D is, for example, “Now we practice the connection and compassion meditation for caring for your partner. You do not need to speak out, but follow my words and talk to the partner in your heart. Let's begin”. The sound of the guidance messages of the compassion meditation is provided a predetermined time after an end of the provision of the sound of voice guidance D. The experiencer performs the compassion meditation according to the guidance messages of the compassion meditation while enjoying the provision of the heartbeat vibration with the intensity according to the interaction and the relaxation music.

The compassion meditation is a meditation in which a person feels connection with himself/herself and other persons and pray for happiness. A person can accept his/her emotions as they are by performing the compassion meditation, enhance his/her gentleness to others, and have the capacity to care about both his/her own happiness and that of others. An effect of the compassion meditation is described, for example, in Non-Patent Literature 1.

The guidance messages of the compassion meditation include a guidance message of compassion meditation for others, a guidance message of compassion meditation for themselves, a guidance message of compassion meditation for himself/herself, and an end message in this order.

An example of the guidance message of the compassion meditation for others is, “May you be happy. May your heart be calm. May you be healthy. May you live at ease. May you be safe”.

An example of the guidance message of the compassion meditation for themselves is, “May you and I, may we be happy. May you and I, may our hearts be at peace. May you and I, may we be healthy. May you and I, may we live with ease. May you and I, may we be safe”.

An example of the guidance message of the compassion meditation for himself/herself is, “May I be happy. May my heart be calm. May I be healthy. May I live at ease. May I be safe”. An example of the end message is, “This is all for the compassion meditation”.

After a predetermined time has elapsed since the start of the sounds of voice guidance D and the guidance message of the compassion meditation, the connection evoking control section 232 ends the provision of the stimuli in the active mode. The connection evoking control section 232 then causes the headphones 162 to provide a sound of voice guidance E for several seconds. Voice guidance E is, for example, “This is the end of the connection experience. Open your eyes slowly”.

After an end of the provision of voice guidance E, the mindfulness and relaxation control section 231 causes the headphones 62 to end the provision of the audio stimuli.

### Configuration Example of Experiencer Information Table

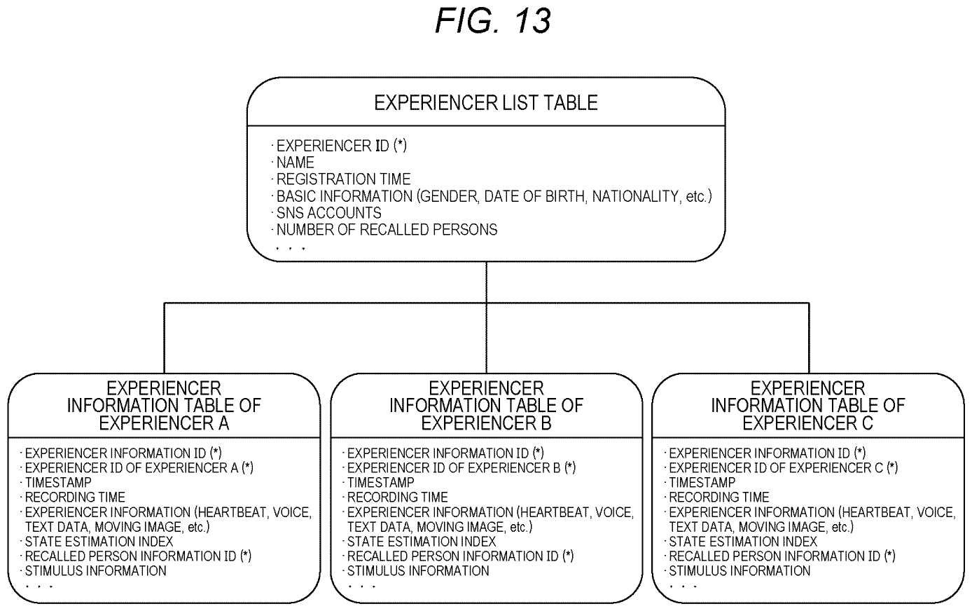

FIG. 13 is a diagram illustrating a configuration example of the experiencer information table stored in the database 130 in FIG. 3.

As illustrated in FIG. 13, the experiencer information table is hierarchically stored in the database 130. Specifically, the database 130 stores an experiencer list table as an upper layer of the experiencer information table. The experiencer list table is a table in which personal information regarding each experiencer is registered in association with an experiencer ID of the experiencer. The personal information includes basic information such as a name, a registration time of the personal information, gender, a date of birth, and nationality, SNS accounts, the number of recalled persons, and the like.

An experiencer table of each experiencer is stored below the experiencer list table for the experiencer. In the example of FIG. 13, there are three experiencers A to C who have used the experience system 10. In this case, experiencer tables of the experiencers A to C are stored below the experiencer list table.

The experiencer ID of the experiencer A is registered in the experiencer table of the experiencer A in association with the experiencer information ID of each piece of experiencer information regarding the experiencer A. The experiencer information ID is an ID unique to each piece of experiencer information. In the experiencer information ID, a timestamp, which is time information at a time when the experiencer information was recorded (generated), a recording time (acquisition time) of the experiencer information, and the experiencer information are registered. This recording time is calculated, for example, on the basis of times clocked by the clocking unit 131 at a start and an end of generation of the experiencer information. A state estimation index corresponding to the experiencer information, a recalled person information ID corresponding to a stimulus provided for the experiencer immediately before acquisition of the experiencer information, and stimulus information regarding the stimulus are also registered in the experiencer information ID of each piece of the experiencer information regarding the experiencer A.

Experiencer tables of the experiencer B and the experiencer C are configured similarly to the experiencer table of the experiencer A except that the experiencer A is replaced with the experiencers B and C, respectively, and description thereof is omitted.

### Example of Information Registered in Experiencer Table

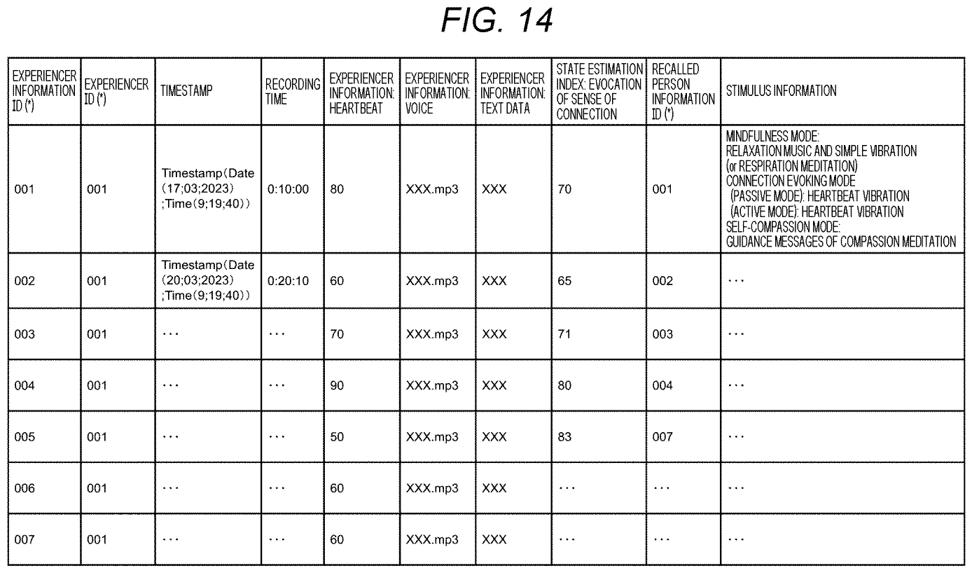

FIG. 14 is a diagram illustrating an example of information registered in the experiencer table of the experiencer A in FIG. 13.

In the example of FIG. 14, a heartbeat, a voice, and text data are generated seven times as the experiencer information regarding the experiencer A. Therefore, the experiencer information table of FIG. 14 includes seven entries. In the example of FIG. 14, for example, experiencer information IDs from “001” to “0007” are added to the seven pieces of experiencer information in order of generation, and each of “001” to “0007” is registered as the experiencer information ID of each entry.

In the example of FIG. 14, the experiencer ID of the experiencer A is “001”, and “001” is registered as the experiencer ID of each entry. In the example of FIG. 14, the experiencer information of the experiencer information ID “001” was generated at 9:19:40 on Mar. 17, 2023. Therefore, in the entry of the experiencer information ID “001”, “Timestamp (Date (17;03;2023); Time (9;19;40)) is registered as a timestamp.

In the example of FIG. 14, the recording time of the experiencer information of the experiencer information ID “001” is 10 seconds, and “0:00:10” is registered as the recording time in the entry of the experiencer information ID “001”.

In the example of FIG. 14, the heartbeat in experiencer information of an experiencer information ID “001” is 80, a file name of the voice is “xxx.mp3”, and the text data is “xxx”. Therefore, in an entry of the experiencer information ID “001”, 80 is registered as the heartbeat of the experiencer information, “xxx.mp3” is registered as the voice, and “xxx” is registered as the text data.

In the example of FIG. 14, the state estimation index indicating a degree of a state of evocation of the sense of connection calculated on the basis of the experiencer information of the experiencer information ID “001” is 70, and 70 is registered in the entry of the experiencer information ID “001” as the state estimation index of the evocation of the Sense of connection.

In the example of FIG. 14, the recalled person information ID of the recalled person information used for a stimulus immediately before the acquisition of the experiencer information of the experiencer information ID “001” is “001”, and “001” is registered as the recalled person information ID in the entry of the experiencer information ID “001”.

In the example of FIG. 14, the stimulus is the stimulus illustrated in FIG. 12. Therefore, in the entry of the experiencer information ID “001”, “relaxation music” and “simple vibration (or respiration mediation)” are registered as the stimulus information in the mindfulness and relaxation mode. In the entry of the experiencer information ID “001”, “heartbeat vibration” is registered as the stimulus information in the passive mode and the active mode of the connection evoking mode, and “guidance message of compassion meditation” is registered as the stimulus information in the self-compassion mode.

Note that although description is omitted, an experiencer ID, a timestamp, a recording time, a heartbeat, a voice, text data, a state estimation index of evocation of a sense of connection, recalled person information ID, and stimulus information are registered in each of entries of experiencer information IDs “002” to “0007”.

Information with “*” at an end in FIGS. 13 and 14 is information that can be referred to from other tables. Specifically, the experiencer IDs of the experiencer list table, and the experiencer information IDs, the experiencer IDs, and the recalled person IDs of the experiencer information tables are information that can be referred to from other tables.

For example, when the experiencer ID “001” registered in the recalled person information table of FIG. 5 is selected, the experiencer information table of FIG. 14 can be referred to. When the recalled person ID “007” registered in the experiencer information table of FIG. 14 is selected, the entry of the recalled person information ID “007” registered in the recalled person information table of FIG. 5 can be referred to.

Types of experiencer information and state estimation index registered in the experiencer information tables are set in advance by the owner of the experience system 10.

### Example of Changes to Stimuli in Mindfulness and Relaxation Mode

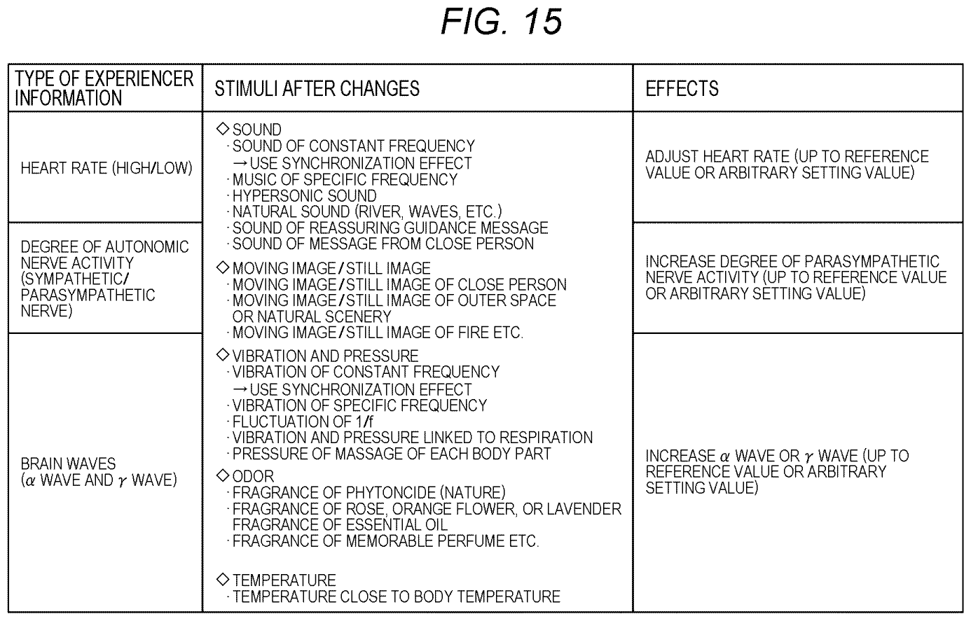

FIG. 15 is a table illustrating an example of changes to the stimuli in the mindfulness and relaxation mode based on the state estimation index.

In a first row from a top of the table in FIG. 15, items “type of experiencer information”, “stimuli after changes”, and “effects” are described. In each of second to fourth rows from the top, information corresponding to the corresponding item is described.

The information corresponding to the item “type of experiencer information” is a type of experiencer information to be used for calculation of the state estimation index used when the mindfulness and relaxation control section 231 determines the stimuli in the mindfulness and relaxation mode. The information corresponding to the item “stimuli after changes” is the stimuli in the mindfulness and relaxation mode changed on the basis of the state estimation index. The item “effects” is target effects of the changes.

Specifically, the information corresponding to the item “type of experiencer information” in the second to fourth rows from the top of the table in FIG. 15 is “heart rate (high and low)”, “degree of autonomic nerve activity (sympathetic nerve/parasympathetic nerve)”, and “brain waves (a wave and Y wave)”, respectively.

That is, the experiencer information to be used for calculation of the state estimation index used when the stimuli in the mindfulness and relaxation mode are determined is the heart rate measured by the heartbeat sensor 155. Alternatively, the experiencer information is a degree of autonomic nerve activity based on the pulse wave measured by the pulse wave sensor 150 and the skin potential measured by the skin potential sensor 160. Alternatively, the experiencer information is brain waves measured by the brain wave sensor 156.

The information corresponding to the items “stimuli after changes” in the second to fourth rows from the top of the table in FIG. 15 is all the same, and is “sound”, “moving image/still image”, “vibration and pressure”, “odor”, and “temperature”.

Specifically, the stimuli after the changes include a sound of a constant frequency for obtaining a synchronization effect provided by the headphones 162, music of a specific frequency, a hypersonic sound, a natural sound of a river, waves, or the like, a sound of a reassuring guidance message, a sound of a message from a close person, and the like.

The stimuli after the changes also include a moving image or a still image of a close person, outer space, natural scenery, fire, or the like provided by the display 168. The stimuli after the changes also include vibration of a constant frequency, vibration of a specific frequency, fluctuation of 1/f, or the like for obtaining the synchronization effect provided by the vibrator 161. The stimuli after the changes include vibration and pressure linked to respiration provided by the vibrator 161 and the pressure providing mechanism 201, pressure of massage provided for each of body parts of the experiencer by the pressure providing mechanism 201, and the like.

The stimuli after the changes also include a fragrance of a phytoncide, rose, orange flower, lavender, essential oil, a memorable perfume, or the like provided by the odor cartridge 164. The stimuli after the changes also include a temperature close to the body temperature provided by the Peltier 165.

It has been found that fragrances of rose, orange flower, and the like promote the secretion of oxytocin. This is described, for example, in Menard “Stem cells get better with aroma (psychological effect)”, retrieved on [Apr. 11, 2023], Internet <URL: https://corp.menard.co.jp/research/tech/tech_05_01.html>. Therefore, the mindfulness and relaxation control section 231 can enhance the oxytocin enhancement loop by causing the odor cartridge 164 to provide a fragrance of rose, orange flower, or the like.

Note that the stimuli after the changes may be a reassuring guidance message provided by the display 168 or a still image or a moving image of a message from a close person.

The information corresponding to the item “effects” in the second to fourth rows from the top of the table in FIG. 15 is “adjust heart rate”, “increase degree of parasympathetic nerve activity”, and “increase α wave and γ wave”, respectively.

That is, the mindfulness and relaxation control section 231 changes the stimuli such that the heart rate becomes a reference value or an arbitrary setting value on the basis of the state estimation index calculated on the basis of the heart rate in the experiencer information. Alternatively, the mindfulness and relaxation control section 231 changes the stimuli on the basis of the state estimation index calculated from the degree of autonomic nerve activity in the experiencer information such that the degree of parasympathetic nerve activity, which indicates a degree of relaxation, reaches a reference value or an arbitrary setting value. Alternatively, the mindfulness and relaxation control section 231 changes the stimuli such that the a wave and the y wave become reference values or arbitrary setting values on the basis of the state estimation index calculated on the basis of the brain waves in the experiencer information.

Note that the mindfulness and relaxation control section 231 may change the stimuli such that the heart rate, the parasympathetic nerve, and the α wave and the γ wave fall within predetermined ranges.

The mindfulness and relaxation control section 231 mindfulness and relaxation control section 231 can also change the stimuli in the mindfulness and relaxation mode on the basis of the state estimation index calculated from the subjective index before and after the provision of the stimuli and during the provision of the stimuli. The mindfulness and relaxation control section 231 can also change the stimuli in the mindfulness and relaxation mode on the basis of the state estimation index calculated from a tone of voice, facial expression, and the like in the experiencer information.

### Description of Transmission Process

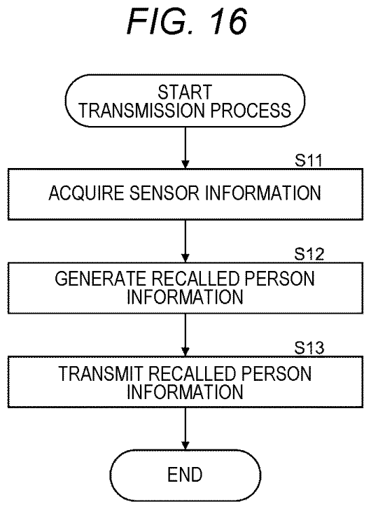

FIG. 16 is a flowchart illustrating a transmission process for transmitting recalled person information by the acquisition device 11.

In step S11 in FIG. 16, the sensor unit 36 acquires sensor information and supplies the acquired sensor information to the recalled person information generation unit 33 via the IF unit 38.

In step S12, the recalled person information generation unit 33 generates the recalled person information on the basis of the sensor information acquired in the processing in step S11. The recalled person information generation unit 33 supplies and stores the recalled person information to and in the storage unit 39. The recalled person information generation unit 33 reads the recalled person information stored in the storage unit 39 and supplies the recalled person information to the communication unit 32 via the IF unit 38.

In step S13, the communication unit 32 transmits the recalled person information generated in the processing in step S12 to the providing device 12, and ends the process.

### Description of Control Process

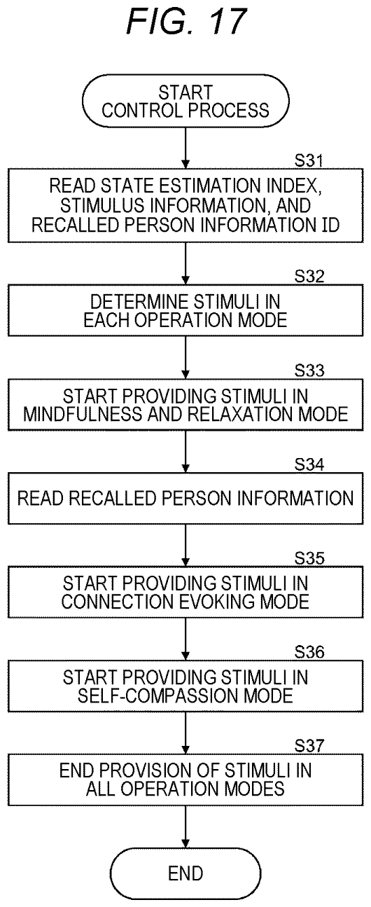

FIG. 17 is a flowchart illustrating a control process for controlling the provision of stimuli by the control unit 124.

In step S31 in FIG. 17, the reading section 230 reads a state estimation index, stimulus information, and a recalled person information ID corresponding to a recalled person ID and an experiencer ID supplied from the IF unit 129 from the experiencer information table stored in the database 130.

The reading section 230 supplies the read state estimation index and stimulus information in the mindfulness and relaxation mode to the mindfulness and relaxation control section 231. The reading section 230 supplies the read state estimation index, stimulus information in the connection evoking mode, and the recalled person information ID to the connection evoking control section 232. The reading section 230 supplies the read state estimation index and stimulus information in the self-compassion mode to the self-compassion control section 233.

In step S32, the control unit 124 determines stimuli in each operation mode. Specifically, the mindfulness and relaxation control section 231 determines the stimuli in the mindfulness and relaxation mode on the basis of the state estimation index read in the processing in step S31 and the stimulus information in the mindfulness and relaxation mode. The control unit 124 supplies stimulus information indicating the stimuli and the experiencer ID to the database 130.

The connection evoking control section 232 determines the stimuli in the connection evoking mode on the basis of the state estimation index read in the processing in step S31, the stimulus information in the connection evoking mode, the recalled person information ID, and the experiencer information supplied from the generation unit 126. The connection evoking control section 232 supplies the database 130 with stimulus information indicating the stimuli, the recalled person information ID of the recalled person information used for the stimuli, and the experiencer ID.

The self-compassion control section 233 determines the stimuli in the self-compassion mode on the basis of the state estimation index read in the processing in step S31 and the stimulus information in the self-compassion mode. The self-compassion control section 233 supplies stimulus information indicating the stimuli and the experiencer ID to the database 130.

In step S33, the mindfulness and relaxation control section 231 starts providing the stimuli in the mindfulness and relaxation mode determination by the processing in step S32. Specifically, the mindfulness and relaxation control section 231 generates stimulus control information for controlling the providing unit 128 in such a way as to provide the stimuli, and supplies the stimulus control information to the providing unit 128 via the IF unit 129.

In step S34, the connection evoking control section 232 reads recalled person information used for the stimuli in the connection evoking mode determination by the processing in step S32 from the recalled person information table of the database 130.

In step S35, the connection evoking control section 232 starts providing the stimuli in the connection evoking mode determined by the processing in step S32 on the basis of the recalled person information read by the processing in step S34. Specifically, the connection evoking control section 232 generates stimulus control information for controlling the providing unit 128 in such a way as to provide the stimuli, and supplies the stimulus control information to the providing unit 128 via the IF unit 129.

In step S36, the self-compassion control section 233 starts providing the stimuli in the self-compassion mode determined by the processing in step S32. Specifically, the self-compassion control section 233 generates stimulus control information for controlling the providing unit 128 in such a way as to provide the stimuli for the user, and supplies the stimulus control information to the providing unit 128 via the IF unit 129.

n step S37, the control unit 124 ends the provision of stimuli in all the operation modes. Specifically, the mindfulness and relaxation control section 231 ends the provision of stimuli started in step S33. The connection evoking control section 232 ends the provision of stimuli started in step S35. The self-compassion control section 233 ends the provision of stimuli started in step S36. The control process then ends.

Note that although the provision of stimuli in all the operation modes is ended in step S37 in the example of FIG. 17, at least a subset of the stimuli in the mindfulness and relaxation mode or the stimuli in the connection evoking mode may be ended at any timing before the processing in step S37.

### Description of Registration Process

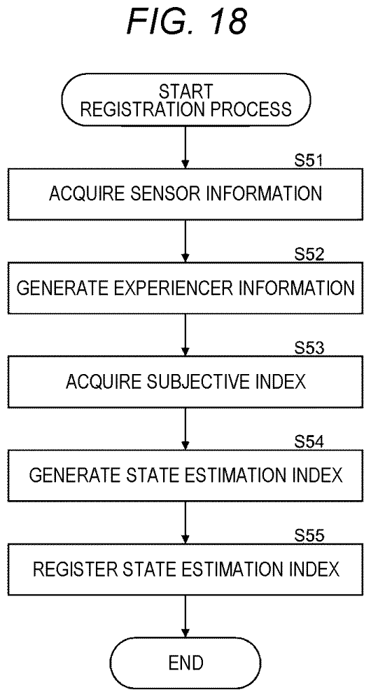

FIG. 18 is a flowchart illustrating a registration process for registering a state estimation index by the providing device 12.

In step S51 in FIG. 18, the sensor unit 127 acquires sensor information and supplies the acquired sensor information to the generation unit 126 via the IF unit 129. In step S52, the generation unit 126 generates the experiencer information on the basis of the sensor information acquired by the processing in step S51, and supplies the experiencer information to the control unit 124, the calculation unit 125, and the database 130.

In step S53, the calculation unit 125 acquires a subjective index received from the smartphone 13 using the communication unit 122 via the IF unit 129. In step S54, the calculation unit 125 generates a state estimation index on the basis of the experiencer information generated in step S52 and the subjective index acquired in step S53.

In step S55, the calculation unit 125 registers the state estimation index generated in the processing in step S54 together with the time information supplied from the clocking unit 131 to the experiencer information table stored in the database 130. The registration process then ends.

Note that the subjective index may be input by the experiencer operating the operation button 143 during the provision of stimuli. In this case, the experiencer presses the operation button 143 the number of times or for a period of time corresponding to the subjective index to input the subjective index at this time.

The amount of oxytocin measured in saliva or the like of the experiencer may be input to the providing device 12, and the providing device 12 may calculate the state estimation index on the basis of the amount of oxytocin. Since oxytocin is directly related to the sense of connection of the experiencer, the providing device 12 can accurately calculate the state estimation index of the evocation of the sense of connection on the basis of the measured amount of oxytocin. Since oxytocin is released by a comfortable pressure (touch) on the skin, the providing device 12 can recognize an effect of a physical stimulus that reproduces a touch of the recalled person on the basis of the measured amount of oxytocin.

The control unit 124 may randomly determine the stimuli in each operation mode without referring to the experiencer information table.

The control unit 124 may generate candidates for stimuli in each operation mode on the basis of the experiencer information table and cause the display 168 or the like to display a plurality of menus including combinations of candidates for stimuli in the operation modes. In this case, the experiencer selects a desired one of the plurality of menus, and the control unit 124 performs control in such a way as to provide stimuli in the operation modes constituting the selected menu. Note that the experiencer may select a desired one of the plurality of menus by specifying conditions.

The recalled person information may be a video of memories of the recalled person and the experiencer or the like. The stimulus information in each operation mode may include a provision time of the stimuli in the operation mode. In this case, the control unit 124 may also change the provision time of the stimuli in each operation mode on the basis of the experiencer information table.

The acquisition device 11 and the providing device 12 may be the same. That is, the recalled person may use a device used when the experiencer enjoys stimuli for the input work of the recalled person information.

The providing device 12 may generate current recalled person information on the basis of the past recalled person information by an AI technology or the like and register the current recalled person information in the database 130. In this case, the experiencer can recall a sense of connection with the recalled person who has already passed away on the basis of the recalled person information.

Sound and images used for the stimuli in the mindfulness and relaxation mode are registered in the database 130 and can be automatically updated at any timing.

The providing device 12 may generate resilience (resistance region) as the experiencer information using, for example, a method described in WO 2022/124085 filed by the present applicant.

If the connection evoking control section 232 performs control such that the stimuli in at least the active mode or the passive mode of the connection evoking mode are provided, it is not necessary to perform control such that the stimuli in both modes are provided.

As described above, the mindfulness and relaxation control section 231 performs control in such a way as to provide stimuli to the experiencer in the mindfulness and relaxation mode, and the connection evoking control section 232 performs control in such a way as to provide stimuli to the experiencer on the basis of the recalled person information in the connection evoking mode. The provision of the stimuli by the mindfulness and relaxation control section 231 is initiated prior to the provision of the stimuli by the connection evoking control section 232. Therefore, it is possible to effectively evoke a sense of connection with the recalled person in the experiencer. As a result, the feeling of loneliness and tension of the experiencer are reduced, and well-being of the experiencer is achieved.

The self-compassion control section 233 performs control in such a way as to provide stimuli to the experiencer in the self-compassion mode, and the stimuli are provided before the stimuli in the connection evoking mode are provided. Therefore, self-compassion can be effectively improved.

As a result, in the end, the experiencer becomes able to strengthen his/her inner mental capacity without relying on external stimuli provided by technology or other scientific means. That is, the experiencer can achieve inner transformation and inner sustainability. As a result, it is possible to contribute to realizing a vibrant life supported by sustained well-being in the era of longevity, where living to 100 years is becoming the norm

As described above, with the experience system 10, it is possible to reduce the feeling of loneliness and tension of the experiencer in a short term. In the medium to long term, a sense of connection with the recalled person can foster a more positive mindset in the experiencer. Enhancing self-compassion can promote personal transformation in the experiencer and help realize inner sustainability. The experiencer can be made to explore the value of happiness based on oxytocin. As a result, it can contribute to the realization of sustained well-being.

On the other hand, many conventional techniques externalize human ability, and do not develop inner non-cognitive ability such as resilience originally possessed by humans. Some of factors of problems of the mind are due to technical progress, and currently the affinity between technical development and improvement of the problems of the mind is not high. For example, development of techniques for attracting attention, such as SNSs, increases stress and a feeling of loneliness in people.

Mindfulness and self-compassion are backed by evidence both academic and scientific, and the effect of the experience system 10 is highly reliable.

Since the control unit 124 determines the stimuli in each operation mode on the basis of the state estimation index, it is possible to select more effective stimuli.

### 2. Computer

The above-described series of processing steps can be performed by hardware or software. In a case where the series of processing steps is performed by software, a program that makes up the software is installed in a computer. Here, examples of the computer include a computer incorporated in dedicated hardware, and for example, a general-purpose personal computer that can execute various functions by installation of various programs.

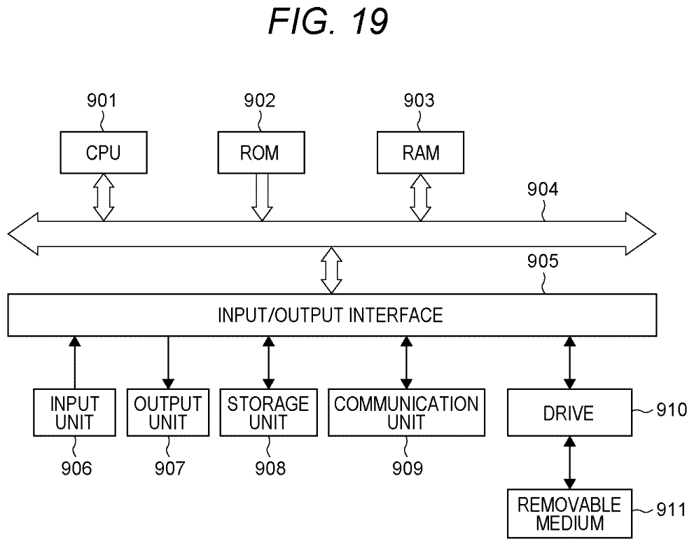

FIG. 19 is a block diagram illustrating an example of configuration of hardware of a computer that executes the above-described series of processing steps by a program.

In the computer, a central processing unit (CPU) 901, a read only memory (ROM) 902, and a random access memory (RAM) 903 are interconnected with a bus 904.

An input/output interface 905 is further connected to the bus 904. An input unit 906, an output unit 907, a storage unit 908, a communication unit 909, and a drive 910 are connected to the input/output interface 905.

The input unit 906 includes a keyboard, a mouse, a microphone, and the like. The output unit 907 includes a display, a speaker and the like. The storage unit 908 includes a hard disk, a non-volatile memory and the like. The communication unit 909 includes a network interface and the like. The drive 910 drives a removable medium 911 such as a magnetic disk, an optical disc, a magneto-optical disk, or a semiconductor memory.

In the computer configured as described above, for example, the CPU 901 loads the program stored in the storage unit 908 into the RAM 903 via the input/output interface 905 and the bus 904 and executes the program, thereby performing the above-described series of processing steps.

The program executed by the computer (CPU 901) can be provided, for example, by being stored in the removable medium 911 as a package medium and the like. Furthermore, the program can be provided via a wired or wireless transmission medium such as a local area network, the Internet, or digital satellite broadcasting.

In the computer, the program can be installed in the storage unit 908 via the input/output interface 905 by mounting the removable medium 911 on the drive 910. Furthermore, the program may be received by the communication unit 909 via a wired or wireless transmission medium to be installed on the storage unit 908. In addition, the program may be installed in advance on the ROM 902 and the storage unit 908.

Note that the program to be executed by the computer may be a program that performs processing in time-series order described in the present specification, or may be a program that performs processing in parallel or at necessary timing such as when a call is made.

Embodiments of the present technology are not limited to the embodiments described above, and various modifications can be made without departing from the scope of the present technology.

For example, it is possible to employ a mode obtained by combining all or some of the plurality of embodiments described above.

For example, the present technology can have a configuration of cloud computing in which one function is shared and processed in cooperation by a plurality of devices via a network.

Furthermore, each step described in the flowchart described above may be performed by a single device, or may be performed by a plurality of devices in a shared manner.

Moreover, in a case where one step includes a plurality of pieces of processing, the plurality of pieces of processing included in the one step can be performed by a one device or performed by a plurality of devices in a shared manner.

Note that the effects described in the present specification are merely examples and are not restrictive, and there may be effects other than those described in the present specification.

The present technology can have the following configurations.

(1)

An information processing device including:
a mindfulness control section that performs control in a mindfulness mode, which is an operation mode for bringing a user into a mindful state, in such a way as to provide a first stimulus for the user; and
a connection evoking control section that performs control in a connection evoking mode, which is an operation mode for evoking a sense of connection in the user, in such a way as to provide a second stimulus for the user on the basis of recalled person information, which is information acquired from a recalled person, who evokes the sense of connection, in which the provision of the first stimulus is initiated prior to the provision of the second stimulus.
   (2)

The information processing device according to (1), in which
the recalled person information is time-series information regarding the recalled person.
   (3)

The information processing device according to (1) or (2), in which
the connection evoking mode includes a passive mode for providing the second stimulus regardless of an input from the user and an active mode for providing the second stimulus according to an input from the user, and
the connection evoking control section performs control in such a way as to provide the second stimulus for the user on the basis of the recalled person information in at least one of the passive mode and the active mode.
   (4)

The information processing device according to any one of (1) to (3), further including:
a self-compassion control section that performs control in a self-compassion mode, which is an operation mode for promoting improvement in self-compassion of the user, in such a way as to provide a third stimulus to the user, in which
the provision of the second stimulus is initiated prior to the provision of the third stimulus.
   (5)

The information processing device according to any one of (1) to (4), further including:
a generation unit that generates feedback information indicating a change in the user due to the first stimulus and the second stimulus.
   (6)

The information processing device according to (5), in which
the generation unit generates the feedback information on the basis of time-series biological information measured by a measurement unit that measures the biological information regarding the user.
   (7)

The information processing device according to (5) or (6), in which
the generation unit generates the feedback information on the basis of an input from the user.
   (8)

The information processing device according to any one of (5) to (7), in which
the mindfulness control section determines the first stimulus to be provided for the user on the basis of the feedback information.
   (9)

The information processing device according to any one of (5) to (8), in which
the connection evoking control section determines the second stimulus to be provided for the user on the basis of the feedback information.
   (10)

The information processing device according to any one of (1) to (9), in which
the second stimulus is a physical stimulus or a cognitive stimulus.
   (11)

An information processing method performed by an information processing device, the information processing method including:
a mindfulness control step of performing control in a mindfulness mode, which is an operation mode for bringing a user into a mindful state, in such a way as to provide a first stimulus for the user; and
a connection evoking control step of performing control in a connection evoking mode, which is an operation mode for evoking a sense of connection in the user, in such a way as to provide a second stimulus for the user on the basis of recalled person information, which is information acquired from a recalled person, who evokes the sense of connection, in which
the provision of the first stimulus is initiated prior to the provision of the second stimulus.
   (12)

A program causing a computer to function as an information processing device including:
a mindfulness control section that performs control in a mindfulness mode, which is an operation mode for bringing a user into a mindful state, in such a way as to provide a first stimulus for the user; and
a connection evoking control section that performs control in a connection evoking mode, which is an operation mode for evoking a sense of connection in the user, in such a way as to provide a second stimulus for the user on the basis of recalled person information, which is information acquired from a recalled person, who evokes the sense of connection, in which the provision of the first stimulus is initiated prior to the provision of the second stimulus.

### REFERENCE SIGNS LIST

12 Providing device
125 Calculation unit
127 Sensor unit
231 Mindfulness control section
232 Connection evoking control section
233 Self-compassion control section

1. An information processing device comprising:
a mindfulness control section that performs control in a mindfulness mode, which is an operation mode for bringing a user into a mindful state, in such a way as to provide a first stimulus for the user; and
a connection evoking control section that performs control in a connection evoking mode, which is an operation mode for evoking a sense of connection in the user, in such a way as to provide a second stimulus for the user on a basis of recalled person information, which is information acquired from a recalled person, who evokes the sense of connection, wherein
the provision of the first stimulus is initiated prior to the provision of the second stimulus.

2. The information processing device according to claim 1, wherein
the recalled person information is time-series information regarding the recalled person.

3. The information processing device according to claim 1, wherein
the connection evoking mode includes a passive mode for providing the second stimulus regardless of an input from the user and an active mode for providing the second stimulus according to an input from the user, and
the connection evoking control section performs control in such a way as to provide the second stimulus for the user on a basis of the recalled person information in at least one of the passive mode and the active mode.

4. The information processing device according to claim 1, further comprising:
a self-compassion control section that performs control in a self-compassion mode, which is an operation mode for promoting improvement in self-compassion of the user, in such a way as to provide a third stimulus to the user, wherein
the provision of the second stimulus is initiated prior to the provision of the third stimulus.

5. The information processing device according to claim 1, further comprising:
a generation unit that generates feedback information indicating a change in the user due to the first stimulus and the second stimulus.

6. The information processing device according to claim 5, wherein
the generation unit generates the feedback information on a basis of time-series biological information measured by a measurement unit that measures the biological information regarding the user.

7. The information processing device according to claim 5, wherein
the generation unit generates the feedback information on a basis of an input from the user.

8. The information processing device according to claim 5, wherein
the mindfulness control section determines the first stimulus to be provided for the user on a basis of the feedback information.

9. The information processing device according to claim 5, wherein
the connection evoking control section determines the second stimulus to be provided for the user on a basis of the feedback information.

10. The information processing device according to claim 1, wherein
the second stimulus is a physical stimulus or a cognitive stimulus.

11. An information processing method performed by an information processing device, the information processing method comprising:
a mindfulness control step of performing control in a mindfulness mode, which is an operation mode for bringing a user into a mindful state, in such a way as to provide a first stimulus for the user; and
a connection evoking control step of performing control in a connection evoking mode, which is an operation mode for evoking a sense of connection in the user, in such a way as to provide a second stimulus for the user on a basis of recalled person information, which is information acquired from a recalled person, who evokes the sense of connection, wherein
the provision of the first stimulus is initiated prior to the provision of the second stimulus.

12. A program causing a computer to function as an information processing device including:
a mindfulness control section that performs control in a mindfulness mode, which is an operation mode for bringing a user into a mindful state, in such a way as to provide a first stimulus for the user; and
a connection evoking control section that performs control in a connection evoking mode, which is an operation mode for evoking a sense of connection in the user, in such a way as to provide a second stimulus for the user on a basis of recalled person information, which is information acquired from a recalled person, who evokes the sense of connection, wherein
the provision of the first stimulus is initiated prior to the provision of the second stimulus.
